(19) United States
(12) Patent Application Publication
(10) Pub. No.: US 2015/0313445 A1
(43) Pub. Date: Nov. 5, 2015

(54) System and Method of Scanning a Body Cavity Using a Multiple Viewing Elements Endoscope

(71) Applicant: EndoChoice, Inc., Alpharetta, GA (US)

(72) Inventors: Tal Davidson, Yokneam Ilit (IL); Yaniv Ofir, Givat Haim Ihud (IL)

(21) Appl. No.: 14/697,933

(22) Filed: Apr. 28, 2015

Related U.S. Application Data
(60) Provisional application No. 62/000,938, filed on May 20, 2014.
(60) Provisional application No. 61/987,021, filed on May 1, 2014.

(51) Int. Cl. A61B 1/00; A61B 1/31; A61B 1/05

(52) U.S. Cl. CPC A61B 1/00009; A61B 1/05; A61B 1/00006; A61B 1/00183; A61B 1/31; A61B 1/00055; A61B 1/00096

(57) ABSTRACT

A method for scanning a body cavity by using an endoscope for capturing images of internal walls of the body cavity is provided. The method includes providing a two dimensional model of the internal walls of the body cavity, the model being used as a reference frame, capturing a sequence of images of the internal walls of the body cavity, associating a plurality of information with at least one of the captured images, and mapping each captured image on a corresponding pre-defined location on the reference frame in real time, in the order of capture, until no portion of the reference frame remains unmapped or until all acquired images are mapped.

### CROSS-REFERENCE TO RELATED APPLICATIONS

The present specification relies on U.S. Patent Application No. 61/987,021, entitled “Real-Time Meta Tagging of Images Generated by A Multiple Viewing Element Endoscope”, and filed on May 1, 2014, for priority.

The present specification also relies on U.S. Patent Application No. 62/000,938, entitled “System and Method for Mapping Endoscopic Images Onto a Reference Frame”, and filed on May 20, 2014, for priority.

The present specification relates to U.S. patent application Ser. No. 14/505,389, entitled “Endoscope with Integrated Sensors”, filed on Oct. 2, 2014, which relies on, for priority, U.S. Provisional Patent Application No. 61/886,572, entitled “Endoscope with Integrated Location Determination”, and filed on Oct. 3, 2013.

The present specification also relates to U.S. Provisional Patent Application No. 62/153,316, entitled “Endoscope with Integrated Measurement of Distance to Objects of Interest”, and filed on Apr. 27, 2015.

All of the above-mentioned applications are herein incorporated by reference in their entirety.

### FIELD

The present invention relates generally to endoscopes, and more specifically, to a method of mapping images of a body cavity captured by an endoscope along with associated information, in real time, during an endoscopic scan, onto a pre-designed model of the body cavity for ensuring completeness of the scan.

### BACKGROUND

An endoscope is a medical instrument used for examining and treating internal body parts such as the alimentary canals, airways, the gastrointestinal system, and other organ systems. Conventionally used endoscopes have at least a flexible tube carrying a fiber optic light guide for directing light from an external light source situated at a proximal end of the tube to a distal tip. Also, most endoscopes are provided with one or more channels, through which medical devices, such as forceps, probes, and other tools, may be passed. Further, during an endoscopic procedure, fluids, such as water, saline, drugs, contrast material, dyes, or emulsifiers are often introduced or evacuated via the flexible tube. A plurality of channels, one each for introduction and suctioning of liquids, may be provided within the flexible tube.

Endoscopes have attained great acceptance within the medical community, since they provide a means for performing procedures with minimal patient trauma, while enabling the physician to view the internal anatomy of the patient. Over the years, numerous endoscopes have been developed and categorized according to specific applications, such as cystoscopy, colonoscopy, laparoscopy, upper gastrointestinal (GI) endoscopy among others. Endoscopes may be inserted into the body's natural orifices or through an incision in the skin.

Endoscopes, that are currently being used, typically have a front camera as well as one or more side cameras for viewing the internal organ, such as the colon, and an illuminator for illuminating the field of view of the camera(s). The camera(s) and illuminators are located in a tip of the endoscope and are used to capture images of the internal walls of the body cavity being endoscopically scanned. The captured images are sent to a control unit coupled with the endoscope via one of the channels present in the flexible tube, for being displayed on a screen coupled with the control unit.

While endoscopes help in detection and cure of a number of diseases in a non-invasive manner, endoscopes suffer from the drawback of having a limited field of view. The field of view is limited by the narrow internal geometry of organs as well as the insertion port, which may be body's natural orifices or an incision in the skin. Further, in order to know the exact position/orientation of an endoscope tip within a body cavity, an operating physician has to usually rely on experience and intuition. The physician may sometimes become disoriented with respect to the location of the endoscope's tip, causing certain regions of the body cavity to be scanned more than once, and certain other regions not being scanned at all.

For the early detection and cure of many diseases such as cancer, it is essential that the body cavity being examined be done in a manner that no region remains un-scanned. Also, the precision of disease detection depends upon a thorough analysis of the images of the internal regions of the body cavity collected during multiple scans separated in time. Sometimes anomalies, such as polyps, may be hidden under folds of the inner linings of the colon, and may not be detected or may be detected insufficiently. While the presence of multiple cameras, including the side cameras that point at different angles than that of the front pointing camera, assists in improving detection of polyps such as those hidden from the view of the front pointing camera; there may still be several instances where the physician may miss viewing the polyps captured by the multiple cameras, by factors such as general oversight or the structure of the colon that may influence the quality of detecting abnormalities. In such cases, missed or delayed detection could result in delayed treatment of diseases like cancer.

Hence, there is need for a method enabling an operating physician to scan a body cavity efficiently without missing any region therein. There is need for a method that ensures an endoscopic scan with a complete and uniform coverage of the body cavity being scanned. There is also need for a method that provides high quality scanning images, of a body cavity being endoscopically scanned, that may be analyzed, tagged, marked and stored for comparisons with corresponding scanned images of the body cavity obtained at a later point in time. There is still further need for a method that also allows verification of an endoscopic examination and double check the presence or absence of disease-causing conditions.

### SUMMARY

The following embodiments and aspects thereof are described and illustrated in conjunction with systems, tools and methods, which are meant to be exemplary and illustrative, not limiting in scope.

The present specification discloses, in some embodiments, a method of scanning a patient's body cavity using an endoscope having multiple viewing elements, wherein the endoscope is controlled by a control unit, the method comprising: selecting a reference frame, having a shape and a scale, from a plurality of reference frames corresponding to the body cavity and to at least one attribute of the patient, wherein the reference frame comprises a plurality of locations corresponding to a plurality of regions of an internal wall of the body cavity; acquiring a plurality of images, each of the plurality of images corresponding to each of the plurality of regions and to each of the plurality of locations on the reference frame, and wherein each of the plurality of images has an associated quality level; and mapping, in real time, each of the plurality of images, corresponding to each of the plurality of locations, on the reference frame, wherein the mapping is done in a sequence in which the plurality of images are acquired.

The present specification also discloses a method of scanning a patient's body cavity using an endoscope having multiple viewing elements, wherein said endoscope is controlled by a control unit, the method comprising: selecting a reference frame, having a shape and a scale, from a plurality of reference frames corresponding to the body cavity and to at least one attribute of the patient, wherein said reference frame comprises a plurality of locations corresponding to a plurality of regions of an internal wall of the body cavity; acquiring a plurality of images, each of said plurality of images corresponding to each of said plurality of regions and to each of said plurality of locations on said reference frame, and wherein each of said plurality of images has an associated quality level; and mapping, in real time, each of said plurality of images, corresponding to each of said plurality of locations, on said reference frame, wherein said mapping is done in a sequence in which said plurality of images are acquired.

The acquisition of said plurality of images may continue until all locations of said plurality of locations of said reference frame are mapped.

Optionally, the method further comprises automatically replacing an image from said plurality of images with an alternative image if an associated quality of said image is lower than an associated quality of said alternative image, wherein said image and said alternative image both correspond to a same region from said plurality of regions.

The associated quality may be defined by a grade selected from values ranging from a first value denoting high quality image to a second value denoting a lower quality image.

The first value denoting high quality image may be associated with an image acquired using a viewing element that has its optical axis oriented at a first angle with respect to the internal wall of the body cavity while the second value denoting a lower quality image may correspond to an image acquired using a viewing element that has its optical axis oriented at a second angle relative to the internal wall of the body cavity, wherein the first angle is closer to 90 degrees than the second angle.

The associated quality may be based on any one or a combination of an angle between the internal wall of the body cavity and an optical axis of a viewing element used to acquire said image, brightness, clarity and contrast of each of said plurality of images.

The attribute may comprise age, gender, weight and body mass index.

The shape of said reference frame may be rectangular.

Optionally, said body cavity is a human colon and said shape of said reference frame approximates a shape of said human colon.

The scale of said reference frame may be 1:10. Optionally, said scale is customizable to a plurality of aspect ratios.

Optionally, the method further comprises marking at least one region of interest in at least one of said plurality of images.

The associated quality may correspond to a specified acceptable quality grade. Optionally, said specified acceptable quality grade varies across said plurality of regions.

The present specification also discloses a method of scanning a patient's body cavity using a tip of a multiple viewing elements endoscope associated with a control unit for executing the method, the method comprising the steps of: automatically recording a start time corresponding to a beginning of an insertion process of said tip within said body cavity; acquiring a plurality of images of an internal wall of said body cavity during said insertion process; identifying at least one anomaly within at least one of said plurality of images; recording and associating a plurality of information with at least one of said plurality of images in real time, wherein at least one of said plurality of information is a first time stamp corresponding to a time taken by said tip to reach proximate said at least one anomaly during said insertion process; automatically recording an end time corresponding to an end of said insertion process and a beginning of a withdrawal process of said tip from said body cavity; automatically recording a second time stamp corresponding to a time elapsed during said withdrawal process; and generating an alert corresponding to said at least one anomaly when said second time stamp is approximately equal to a difference between said end time and said first time stamp.

The at least one anomaly may be identified by a physician by pressing a button on a handle of said endoscope indicating a location of said tip at said at least one anomaly.

Optionally, said plurality of information further comprises at least one of an average color of the internal wall, date and time of said scanning, type, size and anatomical location of said at least one anomaly, type of treatment performed or recommended for future scanning with reference to said at least one anomaly, visual markings to highlight said at least one anomaly, dictation recorded by a physician, and a plurality of patient information including age, gender, weight and body mass index.

Optionally, the method further comprises displaying a bar, said bar being a composite representation of said plurality of images acquired during progress of said insertion process and said plurality of information associated with said at least one of said plurality of images. Optionally, the method further comprises displaying a two dimensional reference frame corresponding to said body cavity.

The present specification also discloses a method of scanning a patient's body cavity using a tip of a multiple viewing elements endoscope associated with a control unit for executing the method, the method comprising the steps of: selecting a reference frame, of a shape and a scale, from a plurality of reference frames corresponding to the body cavity and to at least one attribute of the patient, wherein said reference frame comprises a plurality of locations corresponding to a plurality of regions of an internal wall of the body cavity; automatically recording a start time corresponding to a beginning of an insertion process of said tip within said body cavity; acquiring a plurality of images during said insertion process, each of said plurality of images corresponding to each of said plurality of regions and accordingly to each of said plurality of locations on said reference frame, and wherein each of said plurality of images has an associated quality; identifying at least one anomaly within at least one of said plurality of images; recording and associating a plurality of information with at least one of said plurality of images in real time, wherein at least one of said plurality of information is a first time stamp corresponding to a time taken by said tip to reach proximate said at least one anomaly during said insertion process; mapping, in real time, each of said plurality of images to corresponding each of said plurality of locations on said reference frame, wherein said mapping is done in a sequence in which said plurality of images are acquired and wherein said mapping includes said plurality of information; automatically recording an end time corresponding to an end of said insertion process and a beginning of a withdrawal process of said tip from said body cavity; automatically recording a second time stamp corresponding to a time elapsed during said withdrawal process; and generating an alert corresponding to said at least one anomaly when said second time stamp is approximately equal to a difference between said end time and said first time stamp.

Optionally, the plurality of information further comprises at least one of an average color of the internal wall, date and time of said scanning, type, size and anatomical location of said at least one anomaly, type of treatment performed or recommended for future scanning with reference to said at least one anomaly, visual markings to highlight said at least one anomaly, dictation recorded by a physician, and a plurality of patient information including age, gender, weight and body mass index.

The acquisition of said plurality of images may continue until all locations of said plurality of locations of said reference frame are mapped.

Optionally, the method further comprises automatically replacing an image from said plurality of images with an alternative image if an associated quality of said image is lower than an associated quality of said alternative image, wherein said image and said alternative image both correspond to a same region from said plurality of regions.

The aforementioned and other embodiments of the present specification shall be described in greater depth in the drawings and detailed description provided below.

### BRIEF DESCRIPTION OF THE DRAWINGS

These and other features and advantages of the present specification will be appreciated, as they become better understood by reference to the following detailed description when considered in connection with the accompanying drawings, wherein:

FIG. 1 shows an exploded view of a tip of a multiple viewing elements endoscope according to some embodiments;

FIG. 2A shows a perspective view of the tip of the multiple viewing elements endoscope, of FIG. 1, according to some embodiments;

FIG. 2B shows another perspective view of the tip of the multiple viewing elements endoscope, of FIG. 1, according to some embodiments;

FIG. 3 shows a multiple viewing elements endoscopy system, according to some embodiments;

FIG. 4A illustrates a rectangular reference frame depicting an exemplary pre-designed model of a human colon, in accordance with an embodiment;

FIG. 4B illustrates a shaped reference frame depicting an exemplary pre-designed model of a human colon, in accordance with an embodiment;

FIG. 4C illustrates a diagrammatic representation of a colon having a tubular shape and a corresponding rectangular reference frame, in accordance with an embodiment;

FIG. 4D illustrates a plurality of images of the insides of a colon captured by an endoscope, in accordance with an embodiment;

FIG. 4E illustrates the plurality of images of FIG. 4D mapped onto the reference frame of FIG. 4A, in accordance with an embodiment;

FIG. 4F illustrates measurement of the depth, distance or location of an endoscopic tip using a multiple viewing elements endoscope whose elongated shaft has a plurality of sensors attached thereto, in accordance with an embodiment;

FIG. 4G illustrates an exemplary scenario to demonstrate a method of mapping images of the colon onto the rectangular reference frame of FIG. 4A;

FIG. 4H illustrates the reference frame of FIG. 4E with one of the images therein replaced with a better quality version, in accordance with an embodiment;

FIG. 4I illustrates the reference frame of FIG. 4A overlaid with endoscopic images of a colon, in accordance with an embodiment;

FIG. 5 is a flowchart illustrating a method for scanning a body cavity by using an endoscope, in accordance with an embodiment;

FIG. 6 is a graphical representation of a path of movement of a multiple viewing elements endoscope inside the colon along a time axis, in accordance with some embodiments;

FIG. 7 is a flow chart illustrating an exemplary process of scanning a body cavity and tagging information to a plurality of images captured during the scan;

FIG. 8A illustrates three displays wherein each display corresponds to images collected by one of the viewing elements and wherein a plurality of tagged information and/or notifications appear above or below each image;

FIG. 8B illustrates an embodiment of a color bar and a corresponding reference model display, which appears above or below each image;

FIG. 8C illustrates an exemplary display feature, in one embodiment of the present specification, showing distorted side displays; and

FIG. 8D illustrates another exemplary display feature, in one embodiment of the present specification, showing dimmed side displays.

### DETAILED DESCRIPTION

The present specification is directed towards multiple embodiments. The following disclosure is provided in order to enable a person having ordinary skill in the art to practice the invention. Language used in this specification should not be interpreted as a general disavowal of any one specific embodiment or used to limit the claims beyond the meaning of the terms used therein. The general principles defined herein may be applied to other embodiments and applications without departing from the spirit and scope of the invention. Also, the terminology and phraseology used is for the purpose of describing exemplary embodiments and should not be considered limiting. Thus, the present invention is to be accorded the widest scope encompassing numerous alternatives, modifications and equivalents consistent with the principles and features disclosed. For purpose of clarity, details relating to technical material that is known in the technical fields related to the invention have not been described in detail so as not to unnecessarily obscure the present invention. In the description and claims of the application, each of the words “comprise” “include” and “have”, and forms thereof, are not necessarily limited to members in a list with which the words may be associated.

It is noted that the term “endoscope” as mentioned to herein may refer particularly to a colonoscope and a gastroscope, according to some embodiments, but is not limited only to colonoscopies and/or gastroscopies. The term “endoscope” may refer to any instrument used to examine the interior of a hollow organ or cavity of the body.

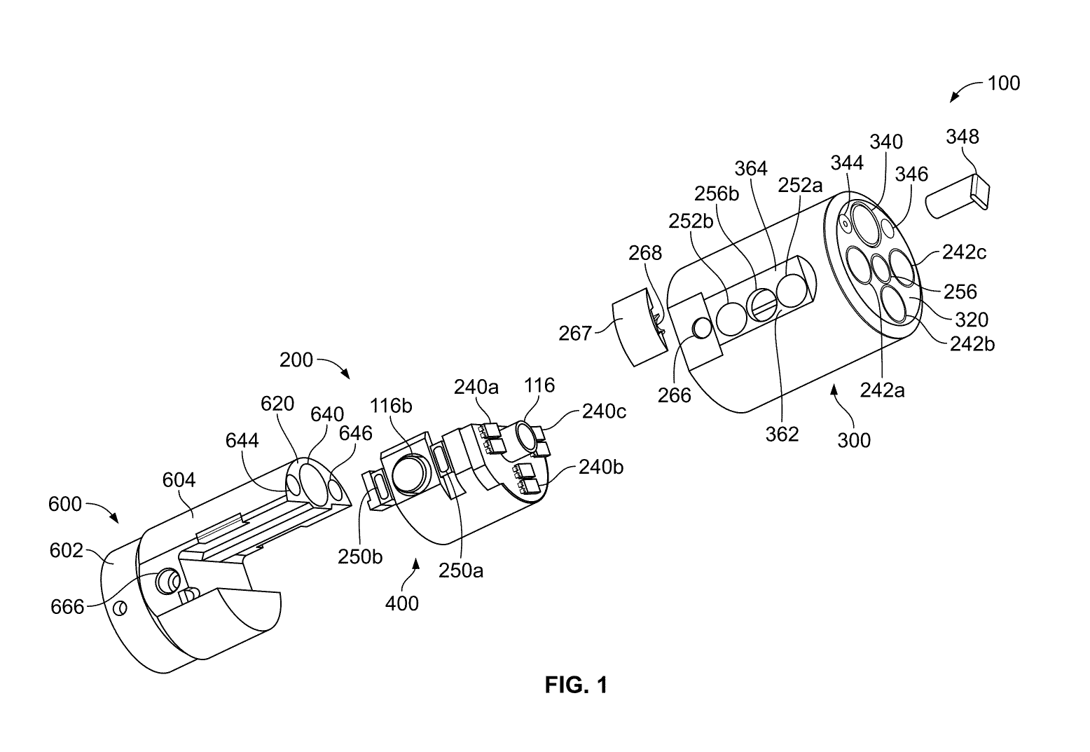

FIG. 1 shows an exploded view of a tip section 200 of a multiple viewing elements endoscope 100 according to an embodiment. In accordance with an embodiment, the tip section 200 of the endoscope 100 includes a tip cover 300, an electronic circuit board assembly 400 and a fluid channeling component 600.

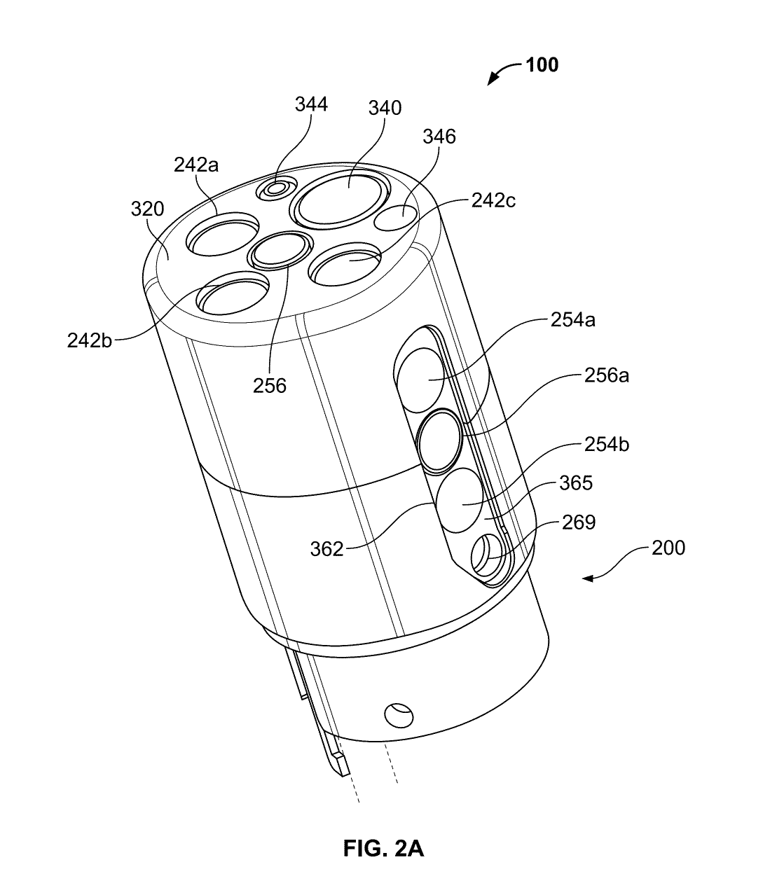

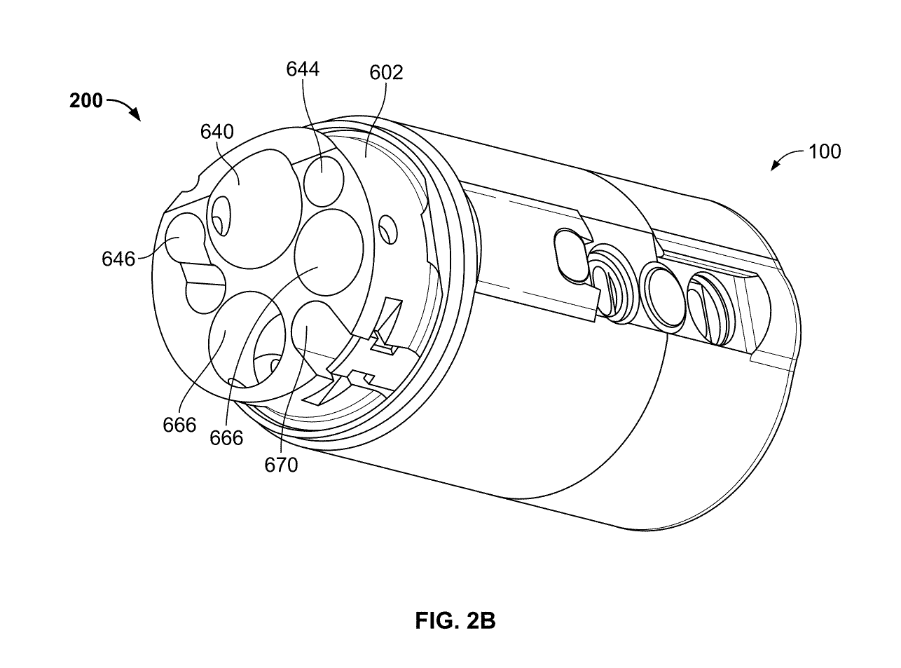

FIGS. 2A and 2B show perspective views of the tip section 200 according to an embodiment. Referring to FIGS. 1, 2A and 2B simultaneously, according to some embodiments, the tip section 200 includes a front panel 320 which comprises four quadrants defined by a vertical axis passing through a center of and lying within a plane of the front panel 320 and a horizontal axis passing through the center and lying within the plane of the front panel 320, wherein the four quadrants include a top left quadrant, a top right quadrant, a bottom left quadrant and a bottom right quadrant. In various embodiments, a transparent surface, window, or opening to front optical lens assembly 256 (of front looking camera or viewing element 116) is positioned on the front panel 320. In various embodiments, a first front optical window 242b, for a first front illuminator 240b, is positioned on the front panel 320, at least partially within the bottom right quadrant and at least partially within the bottom left quadrant. In various embodiments, a second front optical window 242a, for a second front illuminator 240a, is positioned on the front panel 320, at least partially within the bottom left quadrant. In various embodiments, a third front optical window 242c, for a third front illuminator 240c, is positioned on the front panel 320, at least partially within the bottom right quadrant.

In various embodiments, a front working channel opening 340, for front working channel 640, is positioned on the front panel 320, along the vertical axis and at least partially within the top left quadrant and partially within the top right quadrant. In various embodiments, a fluid injector opening 346, for a fluid injector channel 646, is positioned on the front panel 320, at least partially within the top right quadrant. A nozzle cover 348 is configured to fit fluid injector opening 346. In various embodiments, a jet channel opening 344, for a jet channel 644, is positioned on the front panel 320, at least partially within the top left quadrant.

According to some embodiments, fluid channeling component 600 includes a proximal fluid channeling section 602 (or base) which has an essentially cylindrical shape and a unitary distal channeling section 604 (or elongated housing). Distal fluid channeling section 604 partially continues the cylindrical shape of the proximal fluid channeling section 602 in the shape of a partial cylinder (optionally elongated partial cylinder) ending in distal face 620. The distal fluid channeling section 604 has only a fraction of the cylinder (along the height or length axis of the cylinder), wherein another fraction of the cylinder (along the height or length axis of the cylinder) is missing. In other words, in various embodiments, proximal fluid channeling section 602 has a greater width than distal fluid channeling section 604. In various embodiments, the distal fluid channeling section 604 is integrally formed as a unitary block with proximal fluid channeling section 602. The height or length of distal fluid channeling section 604 may by higher or longer than the height or length of proximal fluid channeling section 602. In the embodiment comprising distal fluid channeling section 604, the shape of the partial cylinder (for example, partial cylinder having only a fraction of a cylindrical shape along one side of the height axis) provides a space to accommodate the electronic circuit board assembly 400.

Distal fluid channeling section 604 includes working channel 640, which is configured for insertion of a surgical tool, for example, to remove, treat and/or extract a sample of an object of interest found in a colon or its entirety for biopsy. Distal fluid channeling section 604 further includes the jet fluid channel 644 which is configured for providing a high pressure jet of fluid, such as water or saline, for cleaning the walls of the body cavity (such as the colon) and optionally for suction. Distal fluid channeling section 604 further includes injector channel 646, which is used for injecting fluid (liquid and/or gas) to wash contaminants such as blood, feces and other debris from a surface of front optical lens assembly 256 of forward-looking viewing element 116. Proximal fluid channeling section 602 of fluid channeling component 600 also includes the side injector channels 666 which are connected to side injector openings 266 (on either side of the tip section 200). The proximal fluid channeling section 602 also includes a groove 670 adapted to guide (and optionally hold in place) an electric cable(s) which may be connected at its distal end to the electronic components such as viewing elements (for example, cameras) and/or light sources in the endoscope's tip section 200 and deliver electrical power and/or command signals to the tip section 200 and/or transmit video signals from the cameras to be displayed to a user.

According to some embodiments, fluid channeling component 600 is configured as a separate component from electronic circuit board assembly 400. This configuration is adapted to separate the fluid channels and working channel 640, which are located in fluid channeling component 600 from the sensitive electronic and optical parts that are located in the area of electronic circuit board assembly 400. In some embodiments, the fluid channeling component 600 may include a side working or service channel opening (not shown). Current colonoscopes typically have one working channel opening, which opens at the front distal section of the colonoscope. Such front working channel is adapted for insertion of a surgical tool. The physician is required to perform all necessary medical procedures, such as biopsy, polyp removal and other procedures, through the front opening.

In addition, for treating (removing/biopsying) polyps or lesions found on the side walls of the colon, tip sections that have one or more front working channels only need to be retracted and repositioned with their front facing the polyp or lesion. This re-positioning of the tip may result in “losing” the polyp/lesion and further effort and time must be invested in re-locating it.

Electronic circuit board assembly 400 is configured to carry a front looking viewing element 116, a first side looking viewing element and a second side viewing element 116b which, in accordance with various embodiments, is similar to front looking viewing element 116 and includes a Charge Coupled Device (CCD) or a Complementary Metal Oxide Semiconductor (CMOS) image sensor. The electronic circuit board assembly 400 is configured to carry front illuminators 240a, 240b, 240c, which are associated with front looking viewing element 116 and positioned to essentially illuminate the field of view of front looking viewing element 116.

In addition, electronic circuit board assembly 400 is configured to carry side illuminators 250a and 250b, which are associated with side looking viewing element 116b and positioned to essentially illuminate side looking viewing element's 116b field of view. Electronic circuit board assembly 400 is also be configured to carry side illuminators, which are associated with the opposite side looking viewing element, which may be similar to side illuminators 250a and 250b.

Front illuminators 240a, 240b, 240c and side illuminators 250a and 250b may optionally be discrete illuminators and may include a light-emitting diode (LED), which may be a white light LED, an infrared light LED, a near infrared light LED, an ultraviolet light LED or any other LED.

The term “discrete”, concerning discrete illuminator, may refer to an illumination source, which generates light internally, in contrast to a non-discrete illuminator, which may be, for example, a fiber optic merely transmitting light generated remotely.

Tip cover 300 is configured to fit over the inner parts of the tip section 200 including electronic circuit board assembly 400 and fluid channeling component 600 and to provide protection to the internal components in the inner parts. Front optical lens assembly 256 includes a plurality of lenses, static or movable, which provide a field of view of 90 degrees or more, 120 degrees or more or up to essentially 180 degrees. Front optical lens assembly 256 provides a focal length in the range of about 3 to 100 millimeters. An optical axis of the front looking camera or viewing element 116 is essentially directed along the long dimension of the endoscope. However, since front looking camera or viewing element 116 is typically a wide angle camera, its field of view includes viewing directions at large angles to its optical axis.

Visible on the sidewall 362 of tip cover 300 is a depression 364 wherein placed within depression 364 is a side optical lens assembly 256b for side looking camera or viewing element 116b, which may be similar to front optical lens assembly 256 and optical windows 252a and 252b of illuminators 250a and 250b for side looking camera or viewing element 116b. Also on the sidewall 362 of tip cover 300, on the opposing side to side optical lens assembly 256b, is a depression 365 and an optical lens assembly 256a for another side looking camera, which may be similar to side optical lens assembly 256b, optical windows 254a and 254b for illuminators for a side looking camera or viewing element, and side injector 269. The side optical lens assembly 256b provides a focal length in the range of about 3 to 100 millimeters. In another embodiment, tip section 200 includes only one side viewing element.

It should be appreciated that positioning the side optical lens assembly 256b for side looking camera or viewing element 116b, associated optical windows 252a and 252b of illuminators 250a and 250b and the side injector 266 within the depression 364 prevents tissue damage when cylindrical surface of the tip section 200 contacts a side wall of the body cavity or lumen during an endoscopic procedure. In alternate embodiments, the side viewing element 116b, side illuminators 250a, 250b and side injector 266 may optionally not be located in a depression, but rather be on essentially the same level as the cylindrical surface of the tip section 200.

An optical axis of the first side viewing element 116b is essentially directed perpendicular to the long dimension of the endoscope. An optical axis of the second side viewing element is also essentially directed perpendicular to the long dimension of the endoscope. However, since each side viewing element typically comprises a wide angle camera, its field of view includes viewing directions at large angles to its optical axis. In accordance with some embodiments, each side viewing element has a field of view of 90 degrees or more, 120 degrees or more or up to essentially 180 degrees. In alternative embodiments, optical axis of each side-looking viewing element forms an obtuse angle with optical axis of the front-pointing viewing element 116. In other embodiments, optical axis of each side-looking viewing element forms an acute angle with optical axis of the front-pointing viewing element 116. Thus, it is noted that according to some embodiments, tip section 200 includes more than one side looking viewing elements. In this case, the side looking viewing elements are installed such that their field of views are substantially opposing.

Front-pointing viewing element 116 may be able to detect objects of interest (such as a polyp or another pathology), while side looking viewing element 116b (and/or the second side looking viewing element) may be able to detect additional objects of interest that are normally hidden from front-pointing viewing element 116. Once an object of interest is detected, endoscope operator may desire to insert a surgical tool and remove, treat and/or extract a sample of the polyp or its entirety for biopsy. In some cases, objects of interest may only be visible through only one of front pointing viewing element 116 and one of the two side looking viewing elements.

In addition, side injector opening 266 of side injector channel 666 is located at distal end of sidewall 362. A nozzle cover 267 is configured to fit side injector opening 266.

Additionally, nozzle cover 267 may include a nozzle 268 which is aimed at side optical lens assembly 256b and configured for injecting fluid to wash contaminants such as blood, feces and other debris from a surface of side optical lens assembly 256b of side looking camera or viewing element 116b. The fluid may include gas which may be used for inflating a body cavity. Optionally, nozzle 268 is configured for cleaning both side optical lens assembly 256b and optical windows 252a and/or 252b.

It is noted that according to some embodiments, although tip section 200 is presented herein showing one side thereof, the opposing side may include elements similar to the side elements described herein (for example, side looking camera, side optical lens assembly, injector(s), nozzle(s), illuminator(s), window(s), opening(s) and other elements).

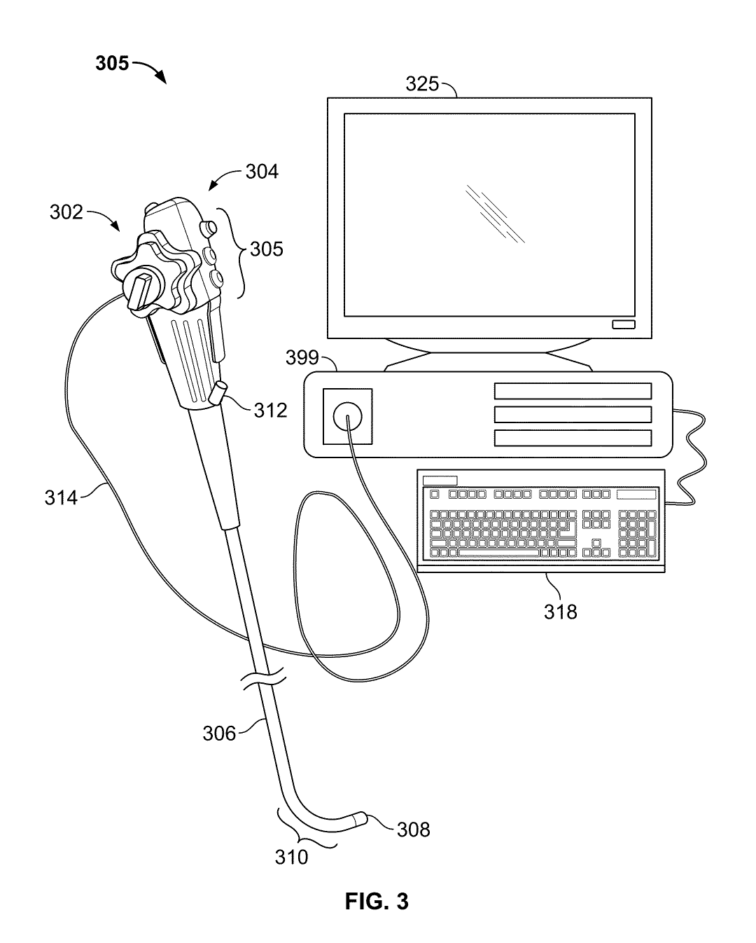

Reference is now made to FIG. 3, which shows a multiple viewing elements endoscopy system 305. System 305 includes a multiple viewing elements endoscope 302. The multiple viewing elements endoscope 302 includes a handle 304, from which an elongated shaft 306 emerges. Elongated shaft 306 terminates with a tip section 308 which is turnable by way of a bending section 310. The handle 304 is used for maneuvering elongated shaft 306 within a body cavity. The handle 304 includes one or more buttons, knobs and/or switches 305 which control bending section 310 as well as functions such as fluid injection and suction. Handle 304 further includes at least one working channel opening 312 through which surgical tools may be inserted as well as one and more side service channel openings.

A utility cable 314, also referred to as an umbilical tube, connects between the handle 304 and a Main Control Unit 399. Utility cable 314 includes therein one or more fluid channels and one or more electrical channels. The electrical channel(s) include at least one data cable for receiving video signals from the front and side-pointing viewing elements, as well as at least one power cable for providing electrical power to the viewing elements and to the discrete illuminators.

The main control unit 399 contains the controls required for displaying the images and/or videos of internal organs captured by the endoscope 302. The main control unit 399 governs power transmission to the endoscope's 302 tip section 308, such as for the tip section's viewing elements and illuminators. The main control unit 399 further controls one or more fluid, liquid and/or suction pump(s) which supply corresponding functionalities to the endoscope 302. One or more input devices 318, such as a keyboard, a touch screen and the like is connected to the main control unit 399 for the purpose of human interaction with the main control unit 399. In the embodiment shown in FIG. 3, the main control unit 399 comprises a screen/display 325 for displaying operation information concerning an endoscopy procedure when the endoscope 302 is in use. The screen 325 is configured to display images and/or video streams received from the viewing elements of the multiple viewing elements endoscope 302. The screen 325 may further be operative to display a user interface for allowing a human operator to set various features of the endoscopy system.

Optionally, the images and/or video streams received from the different viewing elements of the multiple viewing elements endoscope 302 are displayed separately on at least one monitor (not seen) by uploading information from the main control unit 399, either side-by-side or interchangeably (namely, the operator may switch between views from the different viewing elements manually). Alternatively, these images and/or video streams are processed by the main control unit 399 to combine or stitch them into a single, panoramic image or video frame, based on an overlap between fields of view of the viewing elements. In an embodiment, two or more displays are connected to the main control unit 399, each for displaying an image and/or a video stream from a different viewing element of the multiple viewing elements endoscope 302. A plurality of methods of processing and displaying images and/or video streams from viewing elements of a multiple viewing elements endoscope are described in U.S. Provisional Patent Application No. 61/822,563, entitled “Systems and Methods of Displaying a Plurality of Contiguous Images with Minimal Distortion” and filed on May 13, 2013, which is herein incorporated by reference in its entirety.

In an embodiment, the present specification provides a pre-designed model, also referred to as a replica, prototype, representation, mockup or template, of a body cavity or lumen, such as a gastro intestinal (GI) tract, stomach, small intestine, colon, etc., that can be examined by using an endoscope. In an embodiment, the pre-designed model of a body cavity is used as a reference frame for mapping therein images of various parts of the body cavity captured by one or more cameras or viewing elements located in the endoscope tip (such as the tip section 200 of FIG. 1), in real time. The reference frame may be of any shape and dimension depicting the body cavity at a pre-defined yet customizable shape and/or scale. In an embodiment, the reference frame is of rectangular shape. In other embodiments, the reference frame may be of any uniform shape, such as, but not limited to, square, circular, quadrilateral, polygon, that enables efficient analysis of the captured or generated endoscopic images.

In an embodiment, pre-designed models of a plurality of body cavities such as upper GI tract, small intestine, colon, etc., are stored in a memory associated with a control unit of the endoscope and/or a computer system associated with the endoscope. In an embodiment, a plurality of pre-designed models corresponding to a body cavity, differentiable on the basis of at least one of a plurality of characteristics, attributes or parameters, such as age (approximate age or a range encompassing an age), gender, and weight are stored in a database. An operating physician selects a pre-designed model, as a reference frame, corresponding to a desired body cavity of a patient requiring an endoscopic scan and matching, for example, the age and gender of the patient. The selected reference frame is displayed on at least one display screen coupled with the endoscope control unit (such as the Main Control Unit 399 of FIG. 3).

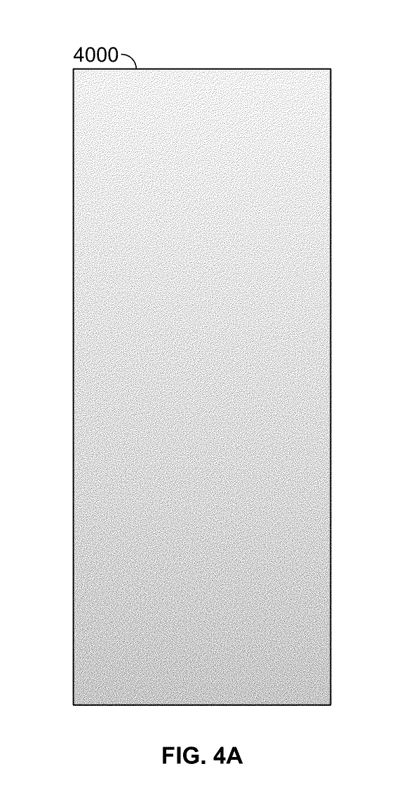

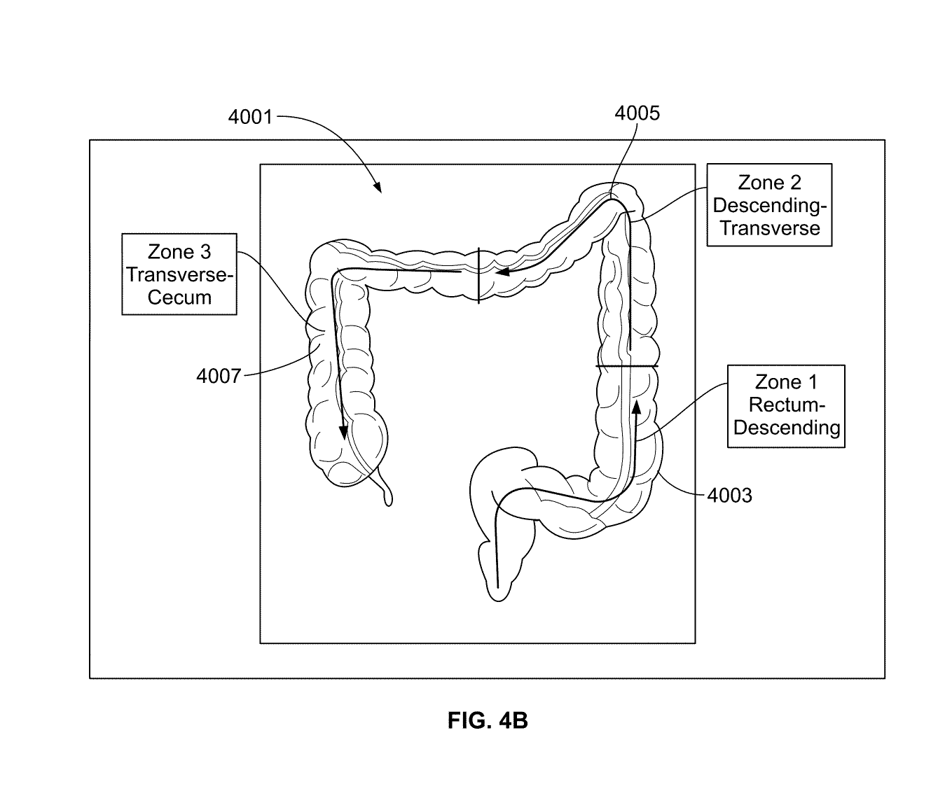

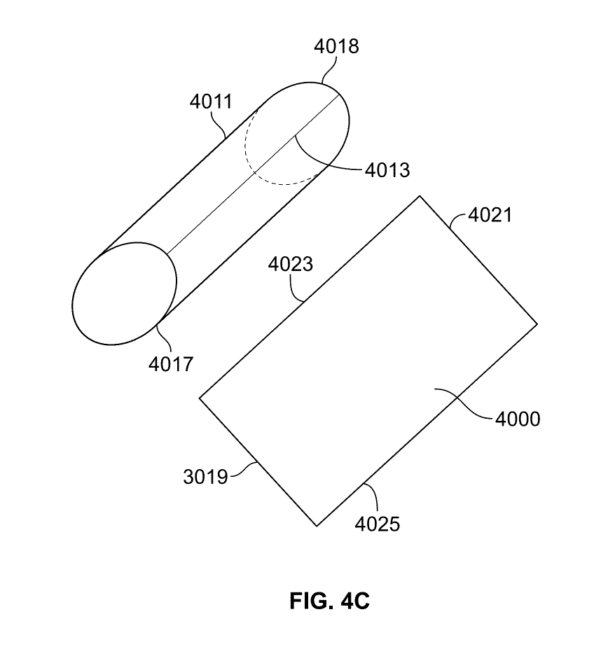

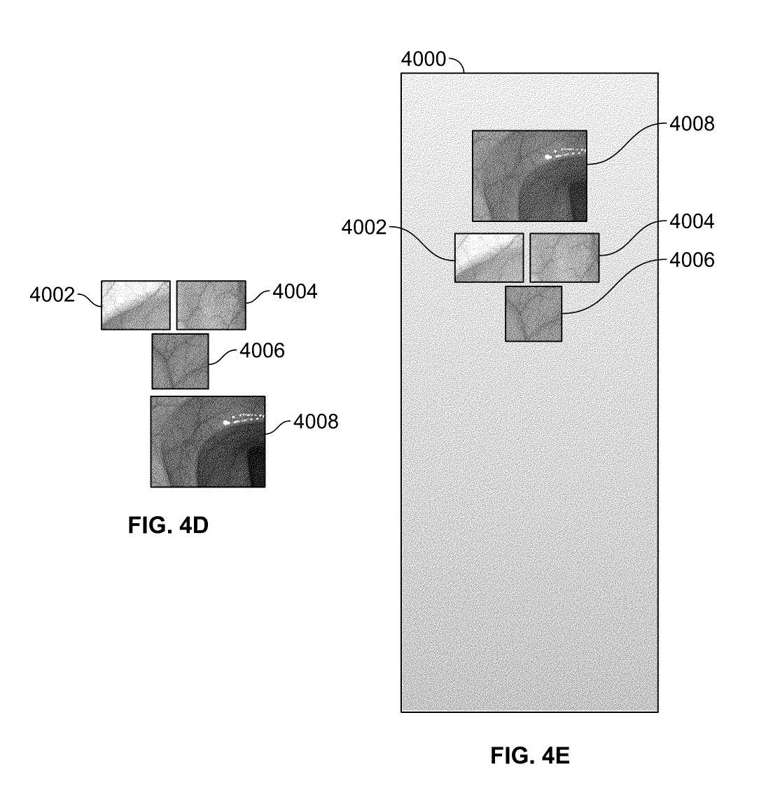

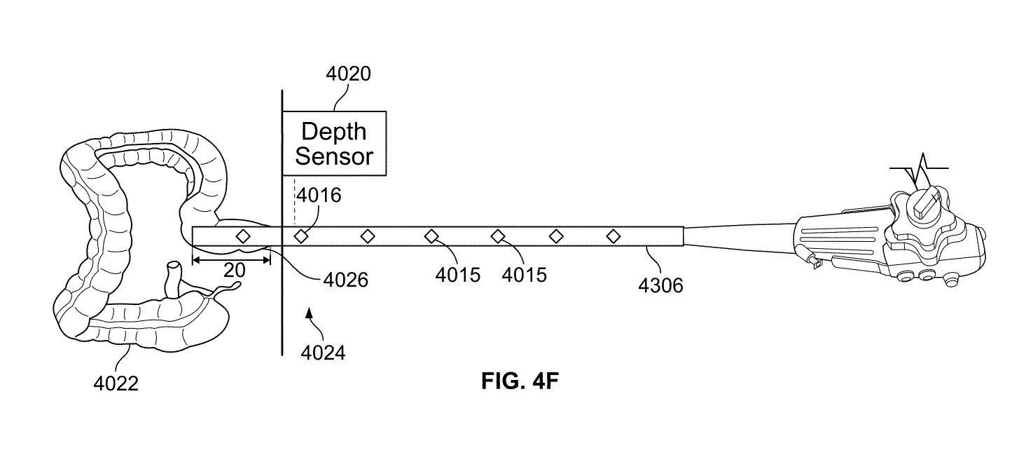

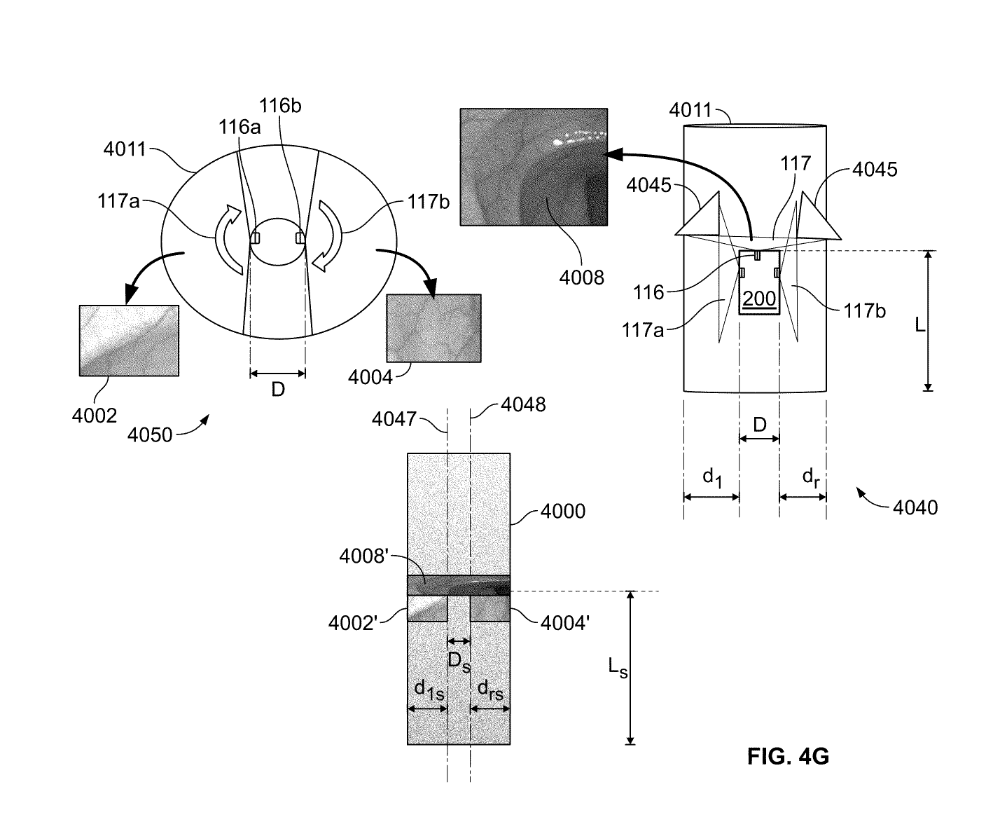

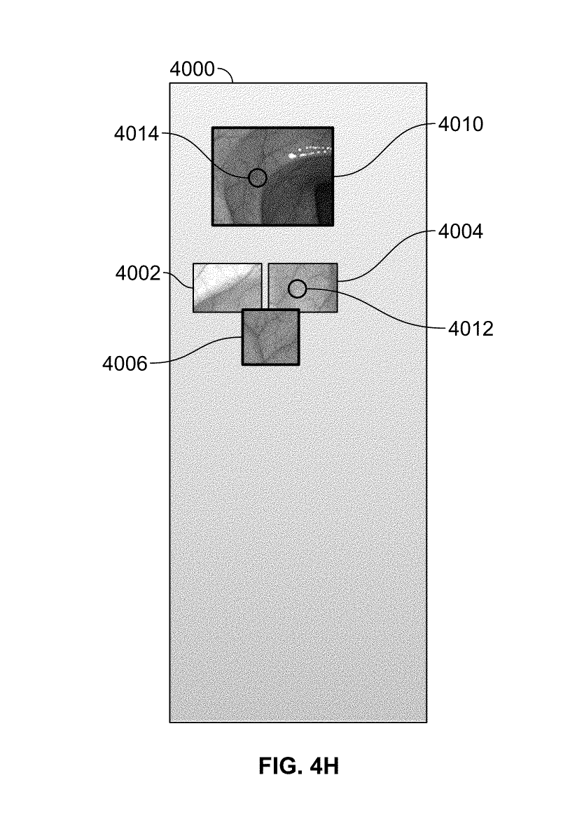

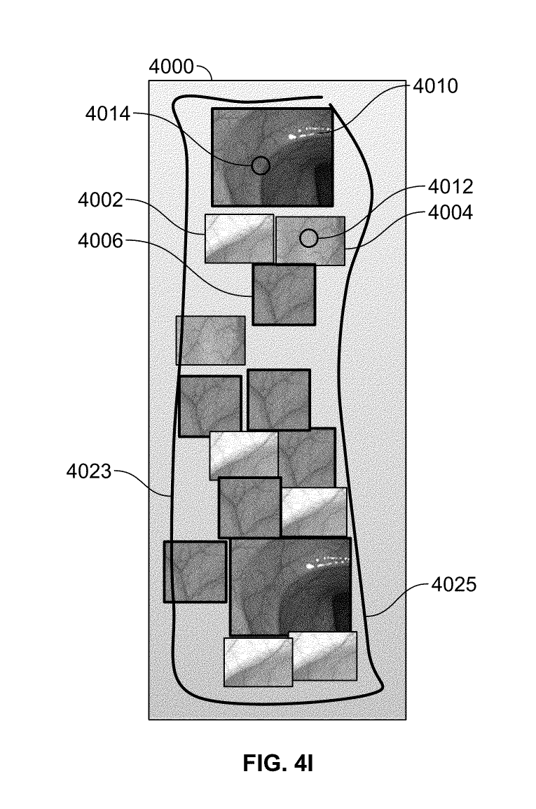

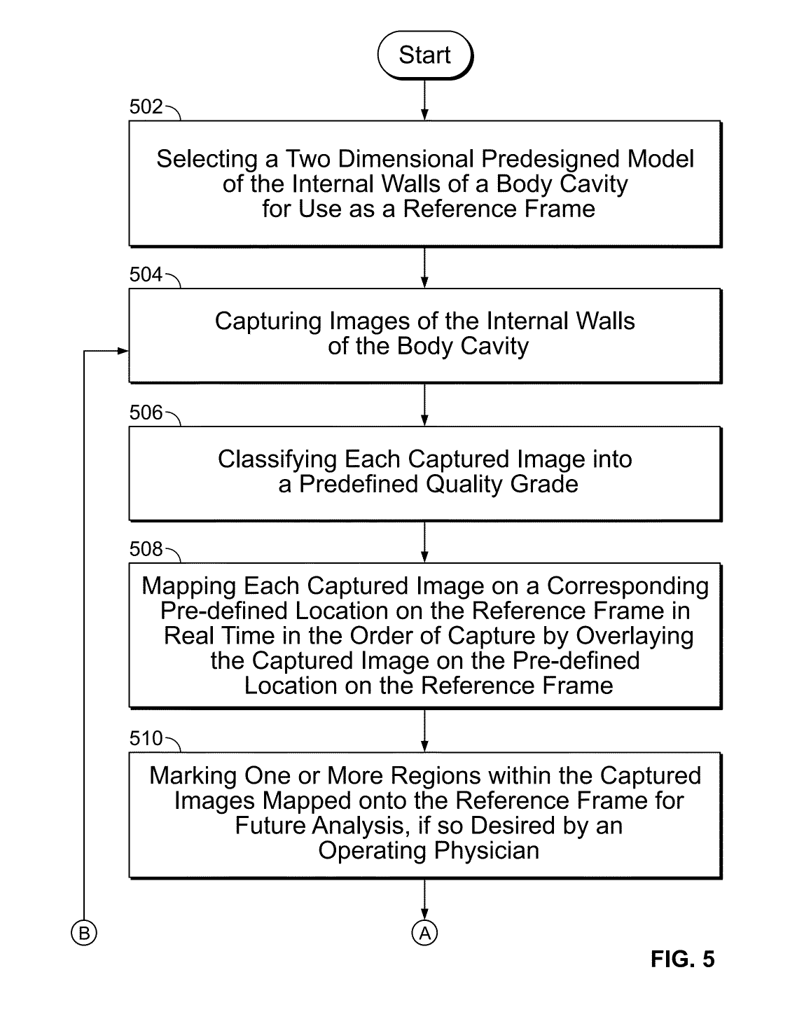

FIG. 4A illustrates a rectangular reference frame, also referred to as a replica, prototype, representation, mockup or template, depicting an exemplary pre-designed model of a human colon, in accordance with an embodiment of the present specification. Reference frame 4000 depicts a pre-designed two dimensional model of a stretched human colon that may be endoscopically examined. In an embodiment, the size of reference frame 4000 is pre-scaled and/or scalable by the physician that is stretched or reduced, to enable viewing the scanned images clearly. In an embodiment, a reference frame depicting scanned images in the scale ratio of 1:10 with respect to the size of the organ or body cavity being scanned is provided by default from the database storing a plurality of pre-designed models. In an embodiment, the dimensions of the reference frame 4000 is customizable, that is scalable up or down, by the physician based on the actual area of the colon to be scanned. Thus, physicians can customize or align the size or scale of the selected reference frame to meet their needs.

FIG. 4B illustrates a shape, such as a ‘C’ shape or a shape approximating the shape of a human colon, reference frame depicting an exemplary pre-designed model of a human colon, in accordance with an embodiment of the present invention. As shown in the figure, reference frame 4001 is shaped as a human colon and depicts a pre-designed two dimensional model of a stretched human colon that may be endoscopically examined. In an embodiment, the reference frame 4001 comprises a first zone 4003 depicting a descending rectum portion, a second zone 4005 depicting a descending transverse portion and a third zone 4007 depicting a transverse cecum portion of a human colon respectively. First zone 4003, second zone 4005, and third zone 4007 of the reference frame 4001 are used for mapping captured endoscopic images of corresponding portions of the actual human colon being scanned. In various embodiments, other shapes of reference frame are available in the database for selection by the physician depending upon the suitability to the endoscopic procedure being carried out. The reference frames advantageously provide a physician with a tool for making sure that no region of the organ, body cavity or lumen being endoscopically examined remains un-scanned. In some embodiments, the tool acts as a checklist for ensuring that the physician has not missed any areas during the scan and for ensuring that the image quality of each millimeter along the colon is adequate for the scan.

FIG. 4C illustrates a diagrammatic representation of a colon having a tubular shape and a rectangular reference frame corresponding to the colon, in accordance with an embodiment of the present specification. In an embodiment, tubular colon 4011 is represented as being transversely cut open along length 4013, in order to obtain a rectangle shaped reference fame 4000 onto which endoscopically obtained images of the internal walls of the colon 4011 are mapped or plotted. The reference frame 4000 represents the “walls” of the tubular colon 4011 when cut along transverse length 4013. Circumferential dimensions 4017 and 4018 of the tubular colon 4011 are represented by sides 4019 and 4021, respectively, of the reference frame 4000, while sides 4023 and 4025 represent the cut edges of length 4013 of the tubular colon 4011. In an embodiment, sides 4023 and 4025 are aligned in order to obtain an accurate representation of the internal walls of the body cavity i.e. the tubular colon 4011 being scanned.

It should be appreciated that as described above with reference to the colon 4011, any other organ, body cavity or lumen is representable on a suitably scaled and shaped corresponding reference frame. A plurality of such scaled and shaped reference frames may be stored in the database for a plurality of corresponding organs, body cavities or lumens. In accordance with an embodiment, the database stores the plurality of such scaled and shaped reference frames or templates in association with a plurality of parameters or characteristics such as age, gender, weight and/or Body Mass Index (BMI).

FIG. 4D illustrates a plurality of images of the inside of a body cavity, such as a colon, captured by one or more viewing elements located in a tip of an endoscope, in accordance with an embodiment of the present invention. Images 4002, 4004, 4006 and 4008 depict different regions of the internal walls of a colon. Referring to FIGS. 4A, 4C, 4D and 4E, in various embodiments, reference frame 4000 is used to map or plot thereon, in real time, the sequentially obtained two dimensional images 4002, 4004, 4006, 4008 of human colon 4011 captured by one or more viewing elements located in a tip of the endoscope scanning the colon.

FIG. 4E illustrates a plurality of images of the insides of a body cavity captured by one or more viewing elements located in an endoscope tip mapped onto a reference frame 4000, in accordance with an embodiment of the present invention. As shown in FIG. 4E, images 4002, 4004, 4006 and 4008 depicting the internal walls of a colon are mapped on to the reference frame 4000 which is a pre-designed model or template of a stretched colon wall. In an embodiment, each image captured by the viewing elements of the scanning endoscope is placed at a corresponding location within the reference frame 4000 by using a mapping method.

Upon completion of the endoscopic scan, in an embodiment, reference frame 4000 is completely covered or overlaid with captured images of every portion of the wall of the colon being scanned and presents a two dimensional image representation of the colon walls. In an embodiment, the method of the present specification enables integrating, combining or stitching together of individual endoscopic images, obtained sequentially, to form a complete two-dimensional panoramic view, illustration or representation of the body cavity being scanned by using the reference frame as a guiding base. A plurality of methods of stitching and displaying images and/or video streams from viewing elements of a multiple viewing elements endoscope are described in U.S. Provisional Patent Application No. 61/822,563, entitled “Systems and Methods of Displaying a Plurality of Contiguous Images with Minimal Distortion” and filed on May 13, 2013, which is herein incorporated by reference in its entirety.

In an embodiment, the mapping method is based on positional parameters such as, but not limited to, the distance between a viewing element of the endoscope and a body tissue being scanned by the viewing element, an image angle, a location, distance or depth of the viewing element (or the tip of the endoscope) within the organ (such as a colon) being scanned, and/or a rotational orientation of the endoscope with respect to the organ or body cavity being scanned. The mapping method, in accordance with an embodiment, utilizes any one or a combination of the positional parameters associated with a scanned image to determine a corresponding location on the shaped and/or scaled reference frame where the scanned image must be mapped or plotted.

In an embodiment, the mapping method is stored as an algorithm in a memory associated with a processor or control unit of the endoscope (such as the Main Control Unit 399 of FIG. 3). Every time an image is captured by the endoscope, the algorithm is executed for comparing the image and its associated positional parameters with portions of the reference frame 4000. The comparison is made by using a set of mapping rules, and the image is placed in a location within the reference frame 4000 that correlates to its relative position in the colon and to the other existing or previously scanned and mapped images. In an embodiment, techniques such as visual feature analysis and stitching algorithms are used to obtain a complete image of a scanned organ by mapping multiple scanned images.

In accordance with an embodiment, the endoscope comprises sensors integrated along its insertion tube to provide real-time information on the distance being travelled by the endoscope inside the patient's lumen. This information is also available on the display associated with the endoscope. This kind of real-time feedback allows the physician to naturally and dynamically determine the location of the endoscope tip and mark any spots with anomalies.

In one embodiment, as shown in FIG. 4F, a plurality of sensors 4015 are placed along the elongated shaft or insertion tube 4306 of the endoscope, also shown earlier as component 306 in FIG. 3. Further, each sensor has a unique identifier, code, signature, or other identification according to its location (such as distance from the distal tip) along the insertion tube 4306. Thus for example, and not limited to such example, a sensor would be placed at a distance of 1, 2, 3, 4, 5, 6, 7, 8, 9, 10, 11, 12, 13, 14, 15, 16, 17, 18, 19, or 20 centimeters, or any increment therein, from the distal end of the tube 4306. The next sensor may be placed at a similar, or different, distance and would have an identifier that is different than the identifier programmed into the first sensor. In another embodiment, each identifier is not only unique to the sensor but also indicative of the particular position, or distance, occupied by the sensor. Thus, in one embodiment, a plurality of sensors are placed at 10 centimeter increments along the length of the insertion tube 4306 where each sensor 4015 has a different identifier and where each identifier is indicative of the distance increment occupied by the sensor. Several different types of sensors may be employed, including, but not limited to inductive sensors, capacitive sensors, capacitive displacement sensors, photoelectric sensors, magnetic sensors, and infrared sensors.

Additionally, a depth sensor is placed at the entrance of the body where the endoscope is inserted and is in communication with the main control unit (such as the unit 399 of FIG. 3) that is used with the endoscope. As a non-limiting example we consider an endoscopic procedure being performed for a patient's colon 4022. The depth sensor 4020 is placed outside the body 4024, close to the rectum 4026, which is the entry point for an endoscope into the colon 4022. In the present example, each sensor is placed 10 cm apart. In operation, the depth sensor 4020 detects alignment to sensor 1205 closest to the entrance site, outside the body. In this case, the closest sensor 4016 which is at a location of 20 centimeters from the tip of the endoscope, and this is indicated by the depth sensor 4020. In one embodiment, each sensor 4015, 4016 is pre-programmed to be read according to its location, such that the 10 cm sensor would transmit a different output than the 20 cm sensor. In one embodiment, the output of the depth sensor 4020 is conveyed to the controller or main control unit, which records and provides a display of the distance travelled by the distal end of the scope.

In some embodiments a matrix of sensors are employed, so that continuity in reading of distances is achieved. In some embodiments touch sensors may be used. Thus, for example, with touch sensors placed at regular intervals on the insertion tube, the number of touch sensors showing an output would indicate the depth the insertion tube has travelled inside the lumen. In one embodiment, the handle (such as the handle 304 of FIG. 3) of the endoscope comprises an actuation device which, when activated, transmits a signal to the processor, controller or main control unit to store a distance measurement corresponding to an endoscopy image. In some embodiments, the actuation device may be a button, switch, touchpad, or any other input device. Thus, whenever an image of the internal walls of the colon 4022 is acquired a corresponding location or distance of the tip of the endoscope is also recorded and associated with the acquired image at that location or distance. In one embodiment, the main control unit is programmed to continuously and automatically acquire images at a predetermined interval of time ‘t’. Thus, in such an embodiment, at every time interval ‘t’ of acquiring an image a corresponding location of the tip is also automatically recorded and associated with the acquired image.

It is known in the art that the insertion tube has numbers or marks on it to indicate to the physician the distance of the insertion tube within patient body. Thus, in another embodiment, an imaging device, such as a CCD, a CMOS and the like, is placed outside the patient's body, close to the entrance point 4026 of the insertion tube 4306 of the endoscope. As shown, for example, the insertion tube 4306 of the endoscope is about 20 cm inside the body. The imaging device captures the “20 cm” mark on the endoscope, and displays the result on an associated display.

In a yet another embodiment, depth is measured by using sensors that respond to the physician's grip on the tube. Sensors are placed over substantially the entire length of the insertion tube, and each sensor has a unique identifier, code, signature, or other identification per its location along elongated axes of the insertion tube. Thus for example, if the physician is holding the tube around the “40 cm” mark, the corresponding sensor at that point responds to the physician's hold, to indicate that the tube is being held at 40 cm. Further, since the typical distance between the point that the physician holds the tube and the body cavity is about 20 cm, this distance can be subtracted from the hold location to obtain an estimate of the depth of the insertion tube inside the body. Thus, in the present example, depth would be 40−20=20 cm, approximately. In one embodiment, an activation device is employed such that the sensors respond only to user's (physician's) hold and activation of sensors on the insertion tube in response to pressure or touch inside the lumen is avoided. Methods and systems of determining the location or distance of an endoscopic tip within a patient's body are described in U.S. patent application Ser. No. 14/505,389, entitled “Endoscope with Integrated Sensors” and filed on Oct. 2, 2014, which is herein incorporated by reference in its entirety.

An exemplary implementation of the mapping method of the present specification will now be discussed with reference to FIG. 4G. Referring now to FIGS. 1, 4F and 4G, in accordance with an exemplary scenario the endoscopic tip 200 is assumed to be at a distance, depth or location ‘L’ within the patient's colon 4011 as shown in a horizontal cross-section view 4040 (in a plane along the length of the colon 4011 and the endoscopic tip 200) and a vertical cross-section view 4050 (in a plane perpendicular to the length of the colon 4011 and the endoscopic tip 200). The tip 200 has a diameter ‘D’, has a front viewing element 116 with a field of view (FOV) 117, a left side viewing element 116a having a FOV 117a and a right side viewing element 116b having a FOV 117b. In accordance with an embodiment, the viewing elements 116, 116a and 116b are equipped with wide FOV lens, ranging from 100 to 180 degrees, thereby providing overlaps 4045. Overlapping field of view regions 4045 are used to measure distances dₗ and dᵣ to the respective left and right walls of the colon 4011. Thus, distances dₗ and dᵣ are calculated using parallax and triangulation techniques, utilizing images from stereo overlap regions 4045 of front and side viewing elements. In the present exemplary scenario, the rotational orientation of the tip 200 is assumed to be such that all the viewing elements 116, 116a and 116b lie in a horizontal plane.

As can be seen from the cross-section views 4040, 4050 the left viewing element 116a captures image 4002 covering area of the left wall (of the colon) equivalent to the FOV 117a, the right viewing element 116b captures image 4004 covering area of the right wall equivalent to the FOV 117b and the front viewing element 116 captures image 4008 covering area of the colon lumen ahead equivalent to the FOV 117. Each of the images 4002, 4004 and 4008 are associated with a plurality of positional parameters, such as the location ‘L’, the respective viewing element that acquired each of the images, the distances d₁ and dᵣ, and the rotational orientation of the tip 200 at the instance of image acquisition.

The images 4002, 4004 and 4008 are now plotted on the two dimensional reference frame or model 4000. The reference frame 4000 is a scaled representation of the colon 4011. For illustration, as an example, the reference frame is rectangular shaped and has an aspect ratio or scale of 1:10. That is, a measurement of 20 cm within the colon 4011 is represented as 2 cm on the scaled reference frame 4000. Thus, in order to plot or map the images 4002, 4004 and 4008 onto the frame 4000 a plurality of mapping rules are implemented, as follows:
The location ‘L ’ of the tip 200 is mapped to a scaled location ‘Lₛ,’ on the reference frame 4000. For example, if the tip is at a distance L=20 cm within the colon 4011 it is determined that the corresponding scaled distance Lₛ=2 cm on the reference frame 4000;
The left and right margins 4047, 4048 on the reference frame 4000 respectively represent the left and right surfaces of the tip 200. The diameter ‘D’ of the tip 200 is scaled to distance ‘Dₛ’ while the left and right distances dₗ and dᵣ are scaled to distances dₗₛ and dᵣₛ on the reference frame 4000.
The image 4002 corresponding to FOV 117a area coverage at a distance dₗ from the left colon wall should be plotted to the left of the left margin 4047 on the reference frame 4000 at the scaled location Lₛ.
The image 4004 corresponding to FOV 117b area coverage at a distance of dr from the right colon wall should be plotted to the right of the right margin 4048 on the reference frame 4000 at the scaled location Lₛ.
The image 4008 corresponding to FOV 117 area coverage in front of the tip 200 should be plotted across the reference frame 4000 covering scaled distances Dₛ, dₗₛ and dᵣₛ at the scaled location Lₛ.
Each of the images 4002, 4004, 4008 are also scaled to plotted images 4002′, 4004′, 4008′ that occupy respective areas on the reference frame 4000 equivalent to the scaled FOVs of the corresponding images.

The reference frame 4000 overlaid with the captured images 4002, 4004, 4006, 4008 as shown in FIG. 4E, is displayed on a screen coupled with the endoscope, enabling the operating physician to visualize the regions of the colon that have been scanned. The operating physician may also see the regions of the reference frame 4000 that are not overlaid by the images depicting the un-scanned portions of the colon, and repeat the scanning process to cover the un-scanned regions as well. By using the reference frame 4000, the physician, in one embodiment, is enabled to scan the entire colon during a single scanning operation without having to repeat the scan at a later time.

In an embodiment, the quality of each image captured by the endoscope is classified by using a grading method. In an embodiment, the grading method comprises classifying each image by comparing one or more aspects of the image, such as the angle between the walls of the organ being examined and an optical axis of the viewing elements being used for the examination, brightness, clarity, contrast, saturation, vibrancy, among other variables, against a predefined set of quality parameters. In an embodiment, an image captured when the optical axis of the viewing element is at a right angle with respect to the wall of the organ being examined by the viewing element is classified as a highest quality grade image, while an image captured when the optical axis of the viewing element is aligned with the organ wall is classified as a lowest grade image.

In an embodiment, the predefined set of quality parameters is stored in a memory coupled with a processor or control unit of the endoscope. In an embodiment, the images are automatically classified into quality grades ranging from 1 to 5 wherein grade 1 denotes a high quality image, while grade 5 denotes a low quality image. Thus, the quality of images decreases from grade 1 to grade 5. In an embodiment, an image of a wall of a body cavity captured by a viewing element whose optical axis is placed perpendicular to the wall (in other words the optical axis of the viewing element is substantially parallel to a normal drawn to the wall), is classified as being of highest or grade 1 quality; whereas if the image is captured by a viewing element whose optical axis is placed obliquely to the wall (or to the normal drawn to the wall) the quality decreases and the image may be classified as grade 2, 3, 4 or 5 depending upon the angle of obliqueness of the capturing viewing element or camera with respect to the wall being imaged.

For example, in one embodiment, where a first image acquired is in focus, is neither overexposed or underexposed, and is taken such that the optical axis of the acquiring viewing element is at an angle of 40 degrees to a normal drawn to the wall (wherein, if the normal drawn is at 90 degrees to the wall, then the angle of orientation of the optical axis with reference to the wall is either 130 or 50 degrees), the quality level assigned to the image is level 2. When a second image is acquired and the second image is in focus, is neither overexposed or underexposed, and is taken such that the optical axis of the acquiring viewing element is at an angle of 20 degrees of normal (that is, the angle of orientation of the optical axis with reference to the wall is either 110 or 70 degrees), the quality level assigned to the image is level 1. In such a case, if both images represent substantially the same area of the body cavity wall, the first image is automatically replaced with the second image, in accordance with an embodiment.

Thus, in accordance with an embodiment, image acquired with the optical axis of the viewing element being at an angle ranging between 0 and 20 degrees with reference to the normal to the wall is assigned a quality grade 1, an optical axis angle range between 21 and 40 degrees with reference to the normal to the wall is assigned a quality grade 2, an optical axis angle range between 41 and 60 degrees with reference to the normal to the wall is assigned a quality grade 3, an optical axis angle range between 61 and 80 degrees with reference to the normal to the wall is assigned a quality grade 4 and an optical axis angle range between 81 and 90 degrees with reference to the normal to the wall is assigned a quality grade 5. The aforementioned optical axis angle ranges are only exemplary and in alternate embodiments different optical axis angle ranges may be assigned different quality grades. For example, a quality grade 1 image may have been acquired with the optical axis of the viewing element being at an angle of 0 to 5 degrees with reference to the normal to the tissue wall, with a quality grade 2 image being acquired at an angle of 6 to 20 degrees, a quality grade 3 image being acquired at an angle of 21 to 50 degrees, a quality grade 4 image being acquired at an angle of 51 to 79 degrees and a quality grade 5 image being acquired at an angle of 80 to 90 degrees. Various other increments, combinations, range can be used.

Also, while in one example only the optical axis angle range is considered as a parameter defining the quality grade, persons of ordinary skill in the art should appreciate that in various alternate embodiments a plurality of parameters may be considered to define the quality grade of an image. For example, parameters such as (but not limited to) underexposure or overexposure (resulting in image artefacts such as saturation or blooming), contrast and degree of in or out of focus along with the optical axis angle with reference to the normal to the wall are each assigned a weightage to generate a weighted resultant parameter. Such weighted resultant parameters are then assigned a quality grade on a reference scale, such as a scale of 1 to 5, a more granular scale of 1 to 10 or a coarser scale of 1 to 3.

In an embodiment, a physician may define or determine the quality grades of images that may be overlaid on the reference frame 4000 before the commencement of an endoscopic scan. The physician may also specify or pre-define the acceptable quality grades of images that may be overlaid on particular regions of the reference frame 4000. For example, since the probability of finding polyps is higher in a transverse colon region as compared to other regions of the colon, the physician may predefine that a region of frame 4000 depicting the transverse colon portion be overlaid only with images of grade 1 quality, whereas the other regions may be overlaid with images of grade 2 quality as well. Thus, the physician defines image quality acceptability standards and may do so prior to commencing the scan operation. In an embodiment, each image mapped onto the reference frame 4000 is automatically graded (by the processor or control unit using the stored set of quality parameters), thereby enabling the operating physician to decide to perform a re-scan for replacing a lower quality grade image with an image having a higher quality grade. FIG. 4H illustrates the reference frame 4000 shown in FIG. 4E with one of the images 4008 therein replaced with a better quality version 4010, in accordance with an embodiment of the present invention.

In an embodiment, the present invention also provides a method for marking, tagging, denoting or annotating one or more regions of the scanned body cavity within the reference frame 4000, in real time, during an endoscopic scan. Marking or annotating regions of interest enables comparison of the marked regions between different endoscopic scans conducted at different times. Such comparisons help in monitoring the progress/commencement of an irregularity or disease within the scanned body cavity. FIG. 4H shows example markings 4012 and 4014 placed over images 3004 and 3010 respectively, by an operating physician, in real time, during the scan. In an embodiment, the operating physician may mark a region in a scanned image being displayed on a screen, by using a pointing accessory, such as a cursor. In an embodiment, the marking comprises a time stamp denoting time taken by the viewing element to reach the tissue portion depicted in the image from a beginning of the scanning procedure. In an embodiment, the marked region is displayed within the reference frame overlaid with a predefined marking symbol, such as circular marks 4012, 4014 shown in FIG. 4H. In another embodiment the operating physician may mark a region of the body cavity being scanned by hitting a foot paddle of a control unit of the endoscope. In an embodiment, each individual image within a pre-designed model serving as a reference frame may be marked, while in another embodiment, regions within each individual frame may also be marked, for analysis at a later time.

In an embodiment, different colors may be used to highlight or mark the boundaries of images captured by the endoscope depicting the angle at which an endoscope's viewing element is placed with respect to a portion of a wall of a body cavity while capturing an image of the portion of the wall. For example, as shown in FIG. 4H, image 4010 which is captured by an obliquely placed camera is marked by a different color boundary as compared to image 4006 which is captured by a perpendicularly placed viewing element with respect to the colon wall being captured. In an embodiment, a user can obtain or perceive the quality grade of an image by comparing the color annotation of the image with a predefined color chart relating each color annotation with a corresponding quality grade.

In various embodiments, a completed endoscopic scan of a body cavity results in a predesigned two dimensional model, reference frame or representation of the body cavity being completely overlaid with images of the body cavity captured by the endoscope. The predesigned model being used as a reference frame ensures that every portion of the body cavity is imaged and each image is placed at a corresponding location on the reference frame, such that no portion of the reference frame remains uncovered by an image. In an embodiment, various image manipulation techniques such as, but not limited to, scaling, cropping, brightness correction, rotation correction, among other techniques, are used to adjust the parameters of an image, before mapping on to the reference frame. Any portion of the reference frame that is not overlaid by a mapped image is indicative of a portion of the body cavity (corresponding to the unmapped area on the reference frame) not being scanned. Hence, the operating physician is prompted to rescan the body cavity until no portion of the reference frame remains uncovered by the mapped endoscopic images.

FIG. 4I illustrates an exemplary reference frame mapped with images of internal walls of a colon scanned by an endoscope, in accordance with an embodiment of the present specification. As shown in FIG. 4I, the reference frame 4000 is overlaid with images of the colon 4002, 4004, 4006, 4010 captured by the endoscope. The unfilled portions of the reference frame 4000 represent corresponding portions of the internal walls of the colon that have not been imaged, thus in this figure, not all colon or body lumen areas have been mapped. In various embodiments, image stitching methods are used to ensure that there are no overlapping images in a reference frame corresponding to a completed endoscopic scan. In an embodiment, lines 4023 and 4025 represent model lines depicting the walls of a tubular body cavity or lumen (such as a colon) being scanned, and are formed such that they accurately represent the relative dimensions of the scanned body cavity.

While the present specification has been described with particular reference to a human colon, it would be apparent to persons of skill in the art that the methods described herein may be used to perform efficient endoscopic scans of any organ, body cavity or lumen.

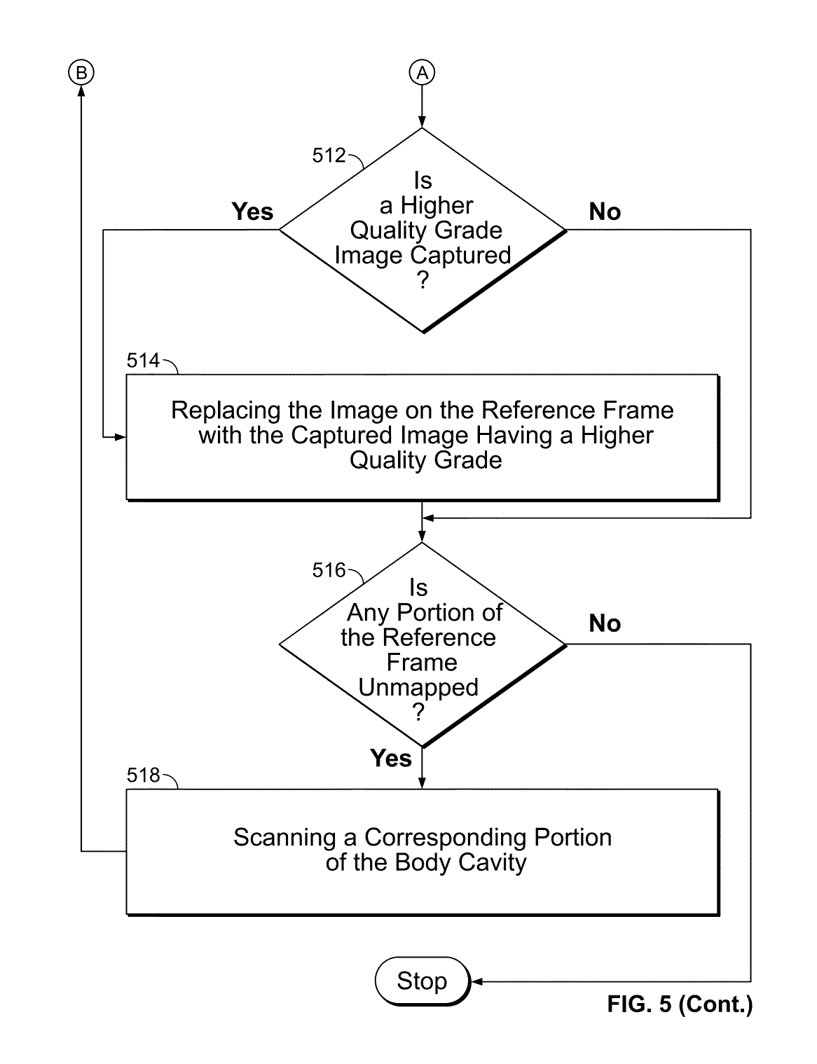

FIG. 5 is a flowchart illustrating a method for scanning a body cavity by using an endoscope, in accordance with an embodiment of the present invention. At step 502 a two dimensional predesigned model of the internal walls of the body cavity for use as a reference frame is provided. In an embodiment, pre-designed models of a plurality of body cavities such as upper GI tract, small intestine, colon, etc., are available in a database stored in a memory associated with a processor or control unit of the endoscope. In an embodiment, the plurality of pre-designed models corresponding to a body cavity, are differentiable on the basis of characteristics such as, but not limited to, age (approximate age or a range encompassing an age), weight, gender and/or BMI. Stated differently, a first pre-designed model accurately represents the relative dimensions and shape of a first person based upon one or more of the first person's age, weight, gender, and/or BMI. A second, different pre-designed model accurately represents the relative dimensions and shape of a second person based upon one or more of the second person's age, weight, gender, and/or BMI. A third pre-designed model accurately represents the relative dimensions and shape of a first person based upon one or more of the first person's age, weight, gender, and/or BMI.

An operating physician selects a pre-designed model corresponding to a desired body cavity of a patient requiring an endoscopic scan and matching at least the age and gender of the patient, as a reference frame for the endoscopic scan. The selected reference frame is displayed on at least one display screen coupled with the endoscope control unit. In various embodiments, the selected reference frame has a shape and scale. The scale of the selected frame is customizable by allowing the operating physician to scale up or down the selected reference frame. In one embodiment, the operating physician can customize the scale of the selected reference frame by simply expanding or shrinking (similar to zooming in or out) the reference frame through a touch screen display and/or by selecting from a plurality of preset scaling or aspect ratio options.

At step 504 the body cavity is scanned and images of the internal walls of the body cavity are captured or acquired. In an embodiment, a plurality of images of the internal walls of the body cavity are captured by using one or more cameras or viewing elements located in a tip of the endoscope (such as the endoscope 100 of FIG. 1).

At step 506 each of the captured images is classified into a quality grade. In an embodiment, the images are classified into quality grades ranging from 1 to 5 wherein grade 1 denotes a highest quality of image, and grade 5 the lowest quality; the quality of images decreasing from grade 1 to grade 5. One of ordinary skill in the art would appreciate that any grading scale can be used, including where the quality scale increases in value with increasing quality and where any range of increments is used therein. In an embodiment, an image of a wall of a body cavity captured by a camera whose optical axis is placed perpendicular to the wall, is classified as being of highest or grade 1 quality; whereas if the image is captured by a camera whose optical axis is placed obliquely to the wall the quality decreases and the image may be classified as grade 2, 3, 4 or 5 depending upon the angle of obliqueness of the capturing camera with respect to the wall being captured.

At step 508 each captured image is mapped or plotted on a corresponding location on the reference frame (selected at step 502), in real time, in the order of capture (or sequentially), by overlaying the captured image on the location on the reference frame. In an embodiment, each image captured by the scanning endoscope cameras is placed at a location within the reference frame by using plurality of positional parameters and mapping rules. In an embodiment, the reference frame overlaid with the captured images is displayed on at least one screen coupled with the endoscope, enabling an operating physician to visualize the regions of the body cavity that have been scanned. The uncovered portions of the reference frame represent corresponding portions of the internal walls of the body cavity that have not been imaged.

At step 510 the operating physician is prompted to mark, tag, annotate or highlight one or more regions within the captured images mapped onto the reference frame, in real time, during the endoscopic scan. Marking regions of interest enables comparison of the marked regions between different endoscopic scans conducted at different times. Such comparisons help in monitoring the progress/commencement of an irregularity or disease within the scanned body cavity. In one embodiment, the operating physician is prompted by automatically displaying a dialog box asking if the physician would like to mark or annotate any portions of the scanned image.

At step 512 it is determined if an image having a higher quality grade as compared to a corresponding image overlaid on the reference frame has been captured. In one embodiment, the processing or control unit displays an alert message to the physician if a new image of a better quality is capture or acquired compared to a previously obtained and mapped image associated with a location on the reference frame. If an image having a higher quality grade is captured, then at step 514 the corresponding image overlaid on the reference frame is replaced with the newly captured image having a higher quality grade.

At step 516 it is determined if any portion of the reference frame is unmapped/uncovered by a captured image. At step 518, if any portion of the reference frame is unmapped/uncovered, endoscopic scan of the corresponding portion of the body cavity is performed and steps 504 to 516 are repeated.

Hence the present specification, in accordance with some aspects, provides a method for conducting an endoscopic scan having a complete coverage of the body cavity being scanned. The endoscopic images captured sequentially are mapped onto corresponding locations of a reference frame which is a predesigned two dimensional model of the stretched body cavity. The mapping ensures that all portions of the body cavity are scanned and none are missed. Further the present specification also provides a method of marking, annotating, tagging or highlighting portions of the scanned images for analysis and comparison with future scanned images. The present specification also provides a method for ensuring collection and record of only a specified quality of scanned images.

The present specification, in accordance with further aspects, describes a method and system of meta-tagging, tagging or annotating real-time video images captured by the multiple viewing elements of an endoscope. In an embodiment, the method and system automatically and continuously captures a plurality of data, information, metadata or metadata tags throughout an endoscopic examination process. In some embodiments, the data that is always automatically captured includes, but is not limited to, the time of the procedure; the location of the distal tip of the endoscope within the lumen or cavity of a patient's body; and/or the color of the body cavity or lumen, such as a colon.

In another embodiment, additional metadata tags are generated and recorded by a press of a button and associated with a frame of video or image data while it is being captured, in real time, such that the physician can revisit these areas with annotated information. The additional metadata includes, but is not limited to, the type of polyp and/or abnormality; the size of the abnormality; and the location of the abnormality.

In another embodiment, additional metadata and variable or additional information is generated and recorded by press of the button and associated with the video or image frame, in real time. Thus, in some embodiments, additional information is captured and/or metadata is created at the video or image frame at which the physician presses the button and includes, but is not limited to, the type of treatment performed and/or recommended treatment for future procedures.

Referring back to the pre-designed model of a human colon of FIG. 4B along with FIG. 3, the elongated shaft or insertion tube 306 of the multiple viewing elements endoscope 302, when inserted into a patient's colon, traverses through a plurality of areas such as the rectum, sigmoid colon, descending colon, transverse colon, ascending colon, and cecum. These plurality of areas of the colon may roughly be divided into three zones, where a first zone 4003 is the distance between the rectum and the descending colon, a second zone 4005 is the distance between the descending colon to the transverse colon, and a third zone 4007 is the distance between the transverse colon and the cecum. Arrows in the figure indicate an exemplary path of the elongated shaft 306 in the direction of insertion inside the colon. During insertion, elongated shaft 306 travels from the first zone 4003 towards third zone 4007 via the second zone 4005. Once the entire colon is traversed, the elongated shaft 306 is withdrawn from the third zone 4007 towards the first zone 4003, followed by its complete withdrawal from the colon by pulling it out.

In embodiments, real time images captured by the viewing elements of the endoscope are viewed on display 325 and/or multiple displays (not shown).

Reference is now made to FIGS. 3, 4B and 5 simultaneously; FIG. 5 is a graphical representation of the path of movement of the elongated shaft 306 inside the colon along a time axis 606, in accordance with some embodiments. Embodiments of the specification allow or enable capturing a plurality of information along with the movement of the elongated shaft 306 of the multiple viewing elements endoscope 302. In various embodiments, the information capture starts at the rectum, at time t(0). Embodiments of the specification may automatically detect the start of an examination and appropriately tag (time stamp) the instance of starting, such as in the form of t(0).

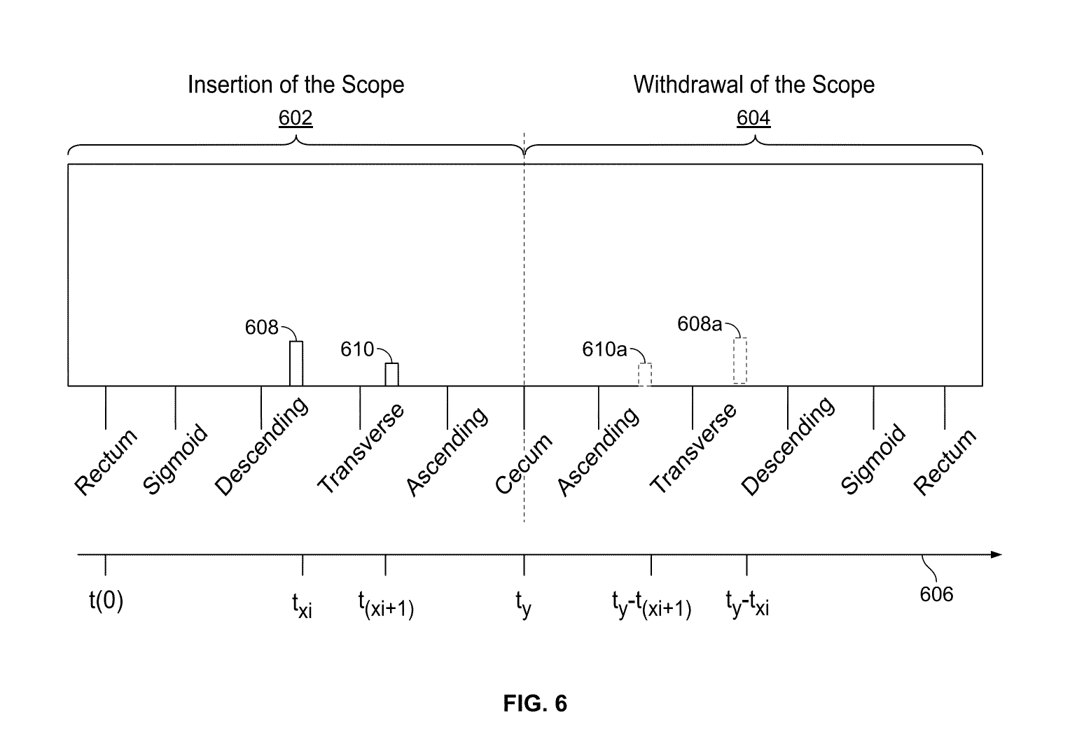

The information may relate to specific instances of areas or regions that have an object of interest, such as an anomaly (for example, a polyp). FIG. 6 illustrates two areas of possible anomaly, 608 captured at a time t(xi) and 610 captured later at a later time t(xi+1), that are detected and recorded throughout the insertion duration 602 of the elongated shaft 306 of the multiple viewing elements endoscope 302. At time ty, the elongated shaft 306 of the endoscope 302 reaches the cecum and thereafter the operator begins to withdraw the scope, in an embodiment.

The anomalies detected during insertion 602 may be detected and recorded once again throughout the withdrawal duration 604 of the elongated shaft 306 of the multiple viewing elements endoscope 302. FIG. 6 illustrates previously captured anomalies as 608a and 610a, during the withdrawal duration 604. Anomaly 610a corresponds to anomaly 610, and is detected first during withdrawal at a time ty=t(xi+1) due to the reverse path of movement of the elongated shaft 306. Similarly, anomaly 608a corresponds to anomaly 608 and is detected at a time ty−t(xi). Thus, in embodiments, the graphical representation of the anomalies is somewhat or approximately symmetrical (with reference to their locations on the time axis 606) as they are captured twice at the same location, however at different times—first during insertion duration 602 and second during withdrawal duration 604.

In various embodiments, the processor or control unit 399 captures a plurality of information, data or metadata automatically. In alternative embodiments, a physician or another operator of the multiple viewing elements endoscope 302 presses a button that captures the information. In embodiments, the button could be located on handle 304, could be a part of the controller 399, or located at any other external location from where the button communicates with the endoscope 302. In other configurations, a voice control can be used to communicate and download information to the controller 399.

Specific instances of information captured with reference to the video streams (generated by the viewing elements of the multiple viewing elements endoscope 302) are thus annotated, tagged or marked by the physician by pressing the button, or otherwise by interfacing with the display 325. An interface, such as a touchscreen may allow the physician to directly highlight, mark, or annotate, in any suitable form, the displayed video images or frames. Thus, embodiments of the specification enable the physician to annotate, highlight, tag or mark a plurality of regions that may be of interest (and should be revisited at a later time), with a plurality of information or metadata, within the captured and displayed image or video frames . Revisiting at a later time, for example during withdrawal duration 604, would further enable the physician to double check and verify anomalies at those locations, if any, treat them and also enable the physician to capture alternative images of the anomalies in case the quality of the previous captured images of the anomalies are found to be below an acceptable level of quality.

In an embodiment, the controller unit 399 automatically and continuously captures a plurality of data throughout the examination process. In some embodiments, the plurality of data that is always automatically captured includes, but is not limited to, the time of start of the procedure; the location of the distal tip within the lumen of the patient's body throughout the endoscopic procedure (such as with reference to the time axis or dimension 606 of FIG. 6); and/or the color of the internal walls of the body cavity or lumen, such as the colon.

In another embodiment, metadata tags are generated and recorded by a press of the button and associated with areas within a frame of video image while it is being captured, in real time, such that the physician can revisit these areas with annotated information. The metadata includes, but is not limited to the type of polyp and/or abnormality; the size of the abnormality; and the location of the abnormality.

Metadata is used herein to refer to data that can be added to images which can provide relevant information to the physician, such as, but not limited to patient information and demographics, time of scan, localization, local average color, physician tags of the image such as type of lesion, comments made, and any additional information that provides additional knowledge about the images.

In another embodiment, additional metadata and variable information is generated and recorded by press of the button and associated with the video or image frame, in real time. Thus, in embodiments, additional information is captured and/or metadata is created at the video frame at which the physician presses the button and includes, but is not limited to the type of treatment performed and/or recommended treatment for future procedures. This is in addition to the information that is automatically and continuously captured using the viewing elements of the endoscope 302. Throughout the insertion duration 602, if the physician sees an anomaly, a button is pressed, the controller unit 399 marks that location, generates metadata, and remembers the location within the examination process where the anomaly was found. The information that is manually collected by the physician can later be compared to the automatically collected data or information.

It should be appreciated that hereinafter the term ‘information’ is used to comprise tagging a video frame or image with any one, a combination or all of: a) automatically and continuously captured plurality of data (by the controller unit 399) such as but is not limited to, the time of start and/or end of the endoscopic procedure, the time location (or time stamp) of the tip of the endoscope within the lumen or cavity of the patient's body, the average color of the body cavity or lumen, such as a colon, date and time of a scan, total time duration of a scan; b) metadata generated and recorded by a press of a button by the physician including, but not limited to, the type of polyp and/or abnormality, the size of the abnormality, the anatomical location of the abnormality; c) additional metadata or variable information such as, but not limited to, the type of treatment performed and/or recommended treatment for future procedures; d) visual markings highlighting at least one area or region of interest related to a captured video or image frame; e) patient information and demographics, such as age, gender, weight, BMI; and f) voice recorded dictation by the physician, such as with reference to an anomaly.

In embodiments, an alert is generated each time an anomaly is detected, such as the anomalies t(xi) and t(xi+1) during the withdrawal duration 604 and/or while repeating an endoscopic procedure at a time different from a previous procedure that had resulted in the detection and tagging of the anomalies. The alert is in the form of a sound, or a color mark or other graphical or visual indication, or any other type of alert that may draw the physician's attention to its presence at the location where it is detected. The location could be time-stamped as illustrated in the graphical representation of FIG. 6, and/or tagged with a plurality of ‘information’ or descriptive within a pictorial representation of the colon such as the reference frame 4001 illustrated in FIG. 4B.

In embodiments, time stamps and graphical or pictorial representations (on a predefined model or a two dimensional reference frame) of anomalies are manifestations of ‘information’ associated with the video streams generated by viewing elements of endoscope 302.

A physician scans the colon on entering and throughout the insertion duration 602 and thereafter treats the detected polyps or any other anomalies throughout the withdrawal duration 604 from the cecum backwards to the rectum, for example. In some embodiments, the physician may treat the polyp during the insertion duration 602. The decision of when to treat a polyp often varies from physician to physician. Embodiments of the specification enable the physician to capture instances of suspected anomalies during insertion 602, and revisit the suspected instances during withdrawal 604 so that they may be re-examined, verified, and treated if confirmed.

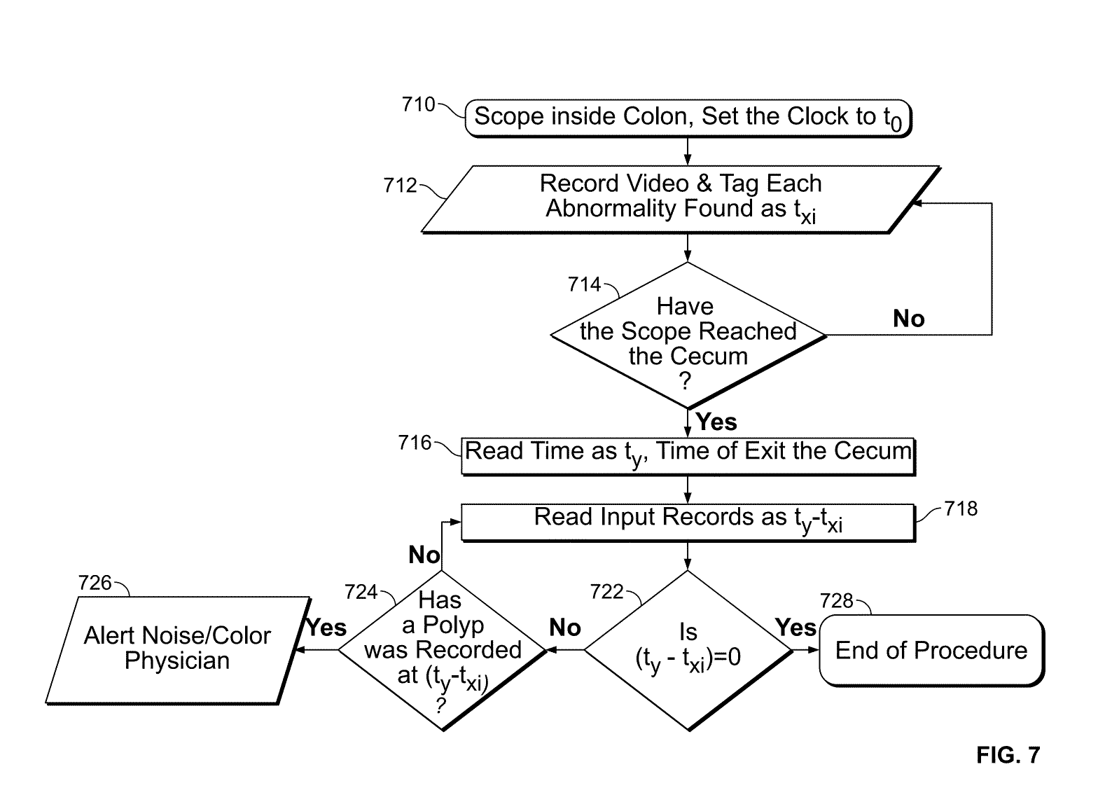

FIG. 7 is a flow chart illustrating an exemplary process of examination and metadata tagging enabled by embodiments of the specification. Referring now to FIGS. 3, 4B, 6 and 7, at 710, the elongated shaft 306, and therefore the tip 308, is inserted inside the colon, and the processor or controller unit 399 starts the timer from t0, marking the initiation of the examination process. At 712, the controller 399 records video streams and, within a recorded video or image frame, enables the physician to tag each detected anomaly (as a result of a press of a button by the physician) as t(xi) based on the continuously auto-detected time location of the endoscopic tip with reference to a time axis or dimension. As described in context of previous figures, optionally the physician tags the suspected anomalies with additional information such as, but not limited to, the type of polyp and/or abnormality, the size of the abnormality, the anatomical location of the abnormality (such as whether within the first, second, third zone 4003, 4005, 4007 shown in FIG. 4B), the type of treatment performed and/or recommended treatment for future procedures, and visual markings to highlight regions of interest. At step 714, it is determined whether the elongated shaft 306 has reached the cecum. If not, it continues to record video streams and tagging detected anomalies, as described at 712, at t(xi). In one embodiment, a time location (or time stamp) of first detected anomaly is denoted by t(xi), a subsequent second anomaly is denoted by t(xi+1), a third anomaly is denoted by t(xi+2) and so on. However, if it is determined that elongated shaft 306 has reached the cecum, at step 716, this time is automatically recorded as ty, which is the time of exit from the cecum. For illustration, it is assumed that during the insertion duration ty, a first anomaly is detected and time stamped or time located as t(xi). Accordingly, during withdrawal of the elongated shaft 306, at step 718, the controller unit 399 continues to record time duration of withdrawal as ty−t(zi) wherein t(zi) is the time taken to reach a location within the colon during withdrawal of the elongated shaft 306. At step 722, it is determined whether ty−t(zi) is zero. A difference of zero would indicate that process of the insertion and the withdrawal is complete, and elongated shaft 306 has returned to the location from where it started. In this case, at 728, the process ends. However, if at 722 it is determined that the difference between ty and t(zi) is positive, the controller unit 399 proceeds to step 724 where it checks whether an anomaly was recorded at an approximate time stamp or time location of ty−t(zi) during the insertion duration. If not, the controller unit 399 continues from step 718. However, if an anomaly was found to be detected at ty−t(zi), at step 626, which means that t(zi) is approximately equal to t(xi) then the controller unit 399 alerts the physician about existence of the anomaly (that is, the first anomaly time stamped or time located at t(xi) during the insertion duration). The physician may respond by verifying and treating the anomaly.

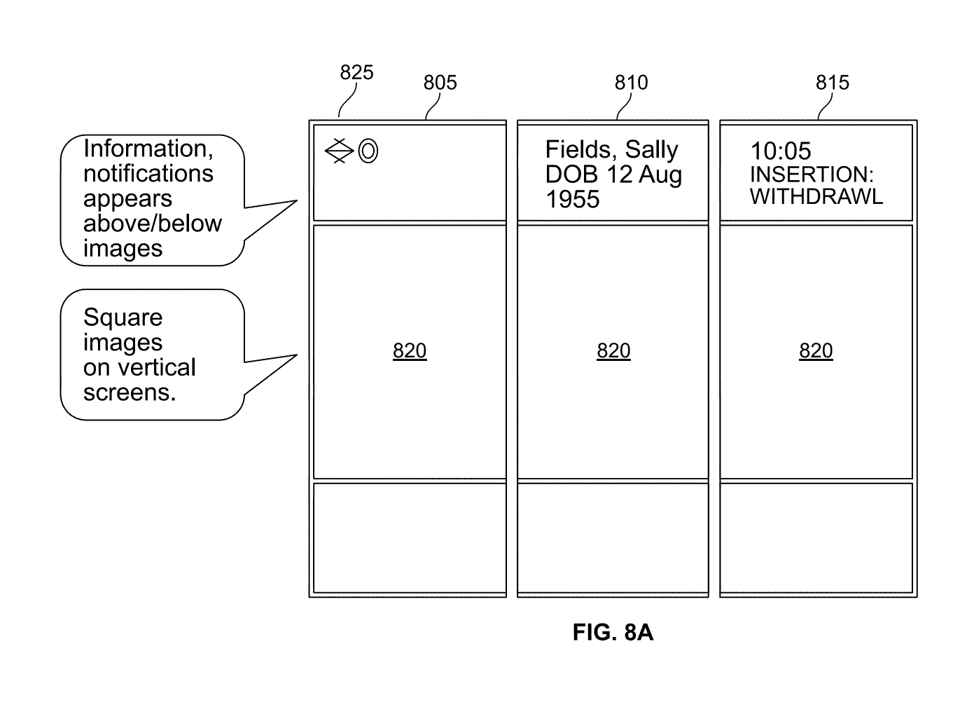

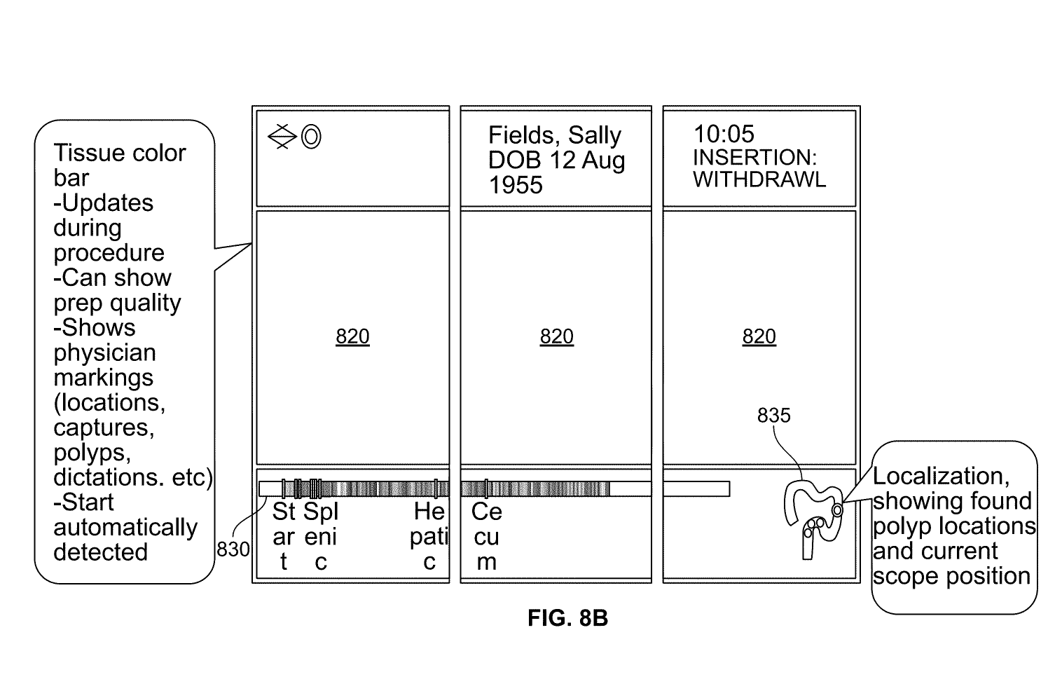

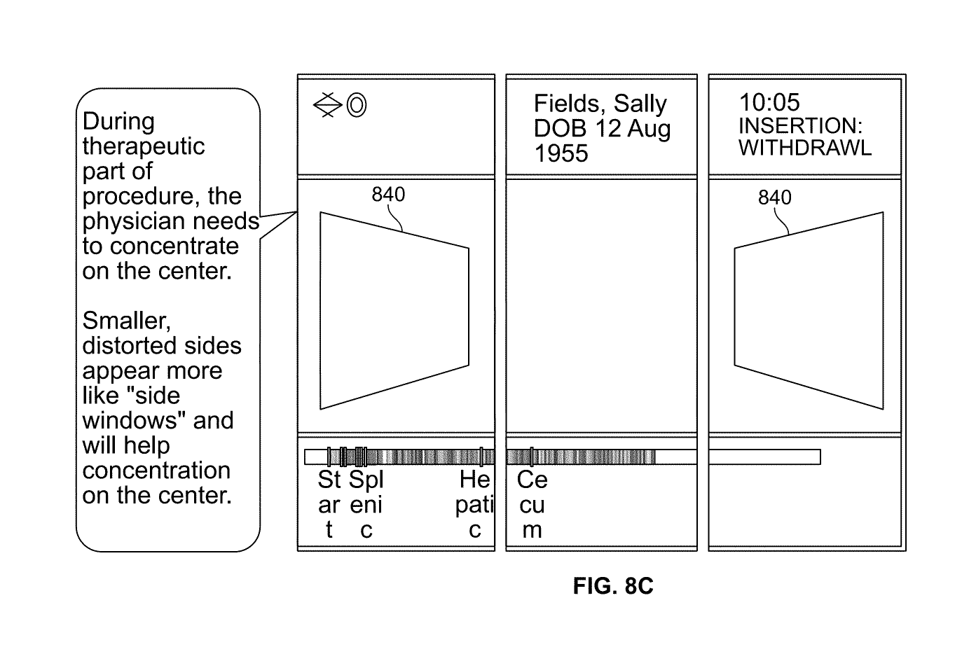

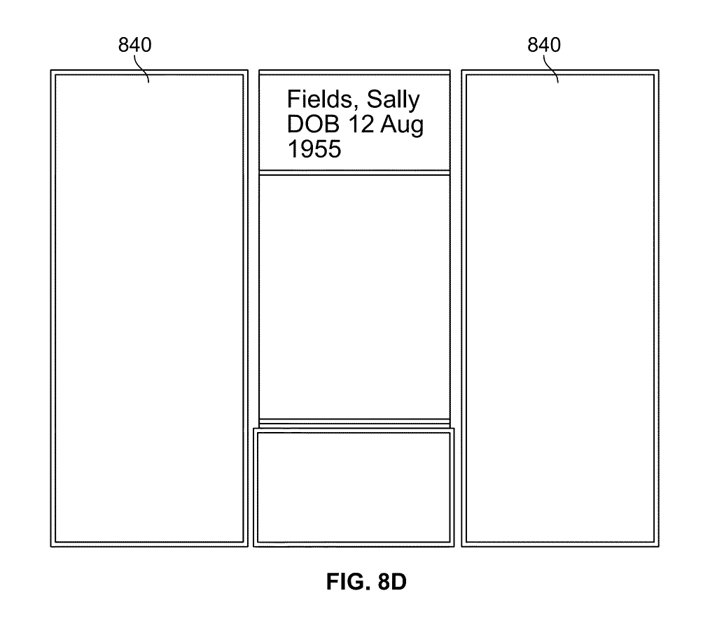

In addition, embodiments of the invention may enable the physician to use the ‘information’ to display or to jump from one tagged location to another in the captured images, using the tags. Further, images and ‘information’ may be used to extract significant instances and observe and compare specific instances. This is further described in context of FIGS. 8A, 8B, 8C, and 8D below.

FIGS. 8A to 8D illustrate exemplary embodiments of one or more displays that are generated by the processor or control unit 399 of FIG. 3. FIG. 8A illustrates three, for example, 9:16 aspect ratio displays 805, 810, and 815 with square images 820. Each display corresponds to images or video frames collected by one of the viewing elements, such as one front and two side viewing elements of the multiple viewing elements endoscope 100 of FIG. 1. In embodiments, a plurality of ‘information’ and/or notifications appear within areas 725 above or below each image 820. Examples of ‘information’ and/or notifications include, but are not limited to, patient information, date of scan, time of scan, total procedure time, whether a video has been recorded, and R (right), C (center), or L (left) to indicate the capturing viewing element.

FIG. 8B illustrates an embodiment of a color bar 830 and a display 835 of a predefined model or two dimensional reference frame of a body cavity (such as the reference frame 4001 of a colon of FIG. 4B), which may appear above or below each image 820. The color bar 730 indicates progress of the examination process in the form of color marks. In embodiments, the color bar 830 is indicative of an average color of the tissues within the internal wall of the body cavity, such as the colon and represents a composite continuum of a plurality of vide frames or images of the internal wall captured during an endoscopic procedure. The color marks are used to better visualize the different areas of the colon and the areas in which abnormalities were found. In addition, the color marks indicate the preparation quality of the colon. For example, before a colonoscopy procedure, the patient is asked to prepare the colon with a special diet. Due to this diet, the colon is expected to be “clean” and free of processed or digested food also referred to as organic dirt. However organic dirt is often found during a colonoscopy procedure and reduces the quality of the resultant images because of a decrease in the visibility of the colon. With decreased visibility in the colon the physician may miss an abnormality resulting in an increase in the rate of missed abnormalities. The physician can use a color bar to indicate a “dirt level” so that the area can be rescanned or examined more carefully during a subsequent procedure. It also indicates the tags, representative of the ‘information’ created by the physician, including locations of the tags, suspected anomalies where the physician pressed the button, any other information added by the physician such as but not limited to dictations. In embodiments, the controller unit automatically detects the initiation of the examination process and starts generating the color bar 830 with the movement of the elongated shaft 306 of FIG. 3. Additionally, the pictorial representation 835 of the body cavity, such as the colon, illustrates the position of the elongated shaft 306 inside the colon, and also the detected and/or suspected anomalies therein.

FIGS. 8C and 8D illustrate exemplary display features, where displays on the sides are manipulated to help focus on an image in the center. In FIG. 8C, the side displays 840 are distorted, such as by reducing the size of their images. In FIG. 8D, the side displays 840 are dimmed or dark-skewed. The display features, illustrated in FIGS. 8C and 8D, enable the physician to reduce the amount of distractions from the side of an image.

Therefore, various embodiments described herein enable the physician to interface with at least one display that provides an overview of an endoscopy or colonoscopy process through the use of multiple viewing elements, in real time. The embodiments also enable the physician to create ‘information’ such as in the form of annotated information; identify locations inside a body cavity, such as the colon, view movement of the scope with respect to time, among other features. The physician may also be able to highlight, annotate, select, expand, or perform any other suitable action on, the displayed images. The physician is therefore able to verify the presence of objects of interest, such as anomalies in the form of polyps, and revisit them with accuracy for treatment.

The above examples are merely illustrative of the many applications of the system of present specification. Although only a few embodiments of the present invention have been described herein, it should be understood that the present invention might be embodied in many other specific forms without departing from the spirit or scope of the invention. Therefore, the present examples and embodiments are to be considered as illustrative and not restrictive, and the invention may be modified within the scope of the appended claims.

We claim:

1. A method of scanning a patient's body cavity using an endoscope having multiple viewing elements, wherein said endoscope is controlled by a control unit, the method comprising:
selecting a reference frame, having a shape and a scale, from a plurality of reference frames corresponding to the body cavity and to at least one attribute of the patient, wherein said reference frame comprises a plurality of locations corresponding to a plurality of regions of an internal wall of the body cavity;
acquiring a plurality of images, each of said plurality of images corresponding to each of said plurality of regions and to each of said plurality of locations on said reference frame, and wherein each of said plurality of images has an associated quality level; and
mapping, in real time, each of said plurality of images, corresponding to each of said plurality of locations, on said reference frame, wherein said mapping is done in a sequence in which said plurality of images are acquired.

2. The method of claim 1, wherein said acquisition of said plurality of images continues until all locations of said plurality of locations of said reference frame are mapped.

3. The method of claim 1, further comprising automatically replacing an image from said plurality of images with an alternative image if an associated quality of said image is lower than an associated quality of said alternative image, wherein said image and said alternative image both correspond to a same region from said plurality of regions.

4. The method of claim 1, wherein said associated quality is defined by a grade selected from values ranging from a first value denoting high quality image to a second value denoting a lower quality image.

5. The method of claim 4, wherein the first value denoting high quality image is associated with an image acquired using a viewing element that has its optical axis oriented at a first angle with respect to the internal wall of the body cavity while the second value denoting a lower quality image corresponds to an image acquired using a viewing element that has its optical axis oriented at a second angle relative to the internal wall of the body cavity, wherein the first angle is closer to 90 degrees than the second angle.

6. The method of claim 1, wherein said associated quality is based on any one or a combination of an angle between the internal wall of the body cavity and an optical axis of a viewing element used to acquire said image, brightness, clarity and contrast of each of said plurality of images.

7. The method of claim 1, wherein said attribute comprises age, gender, weight and body mass index.

8. The method of claim 1, wherein said shape of said reference frame is rectangular.

9. The method of claim 1, wherein said body cavity is a human colon and said shape of said reference frame approximates a shape of said human colon.

10. The method of claim 1, wherein said scale of said reference frame is 1:10.

11. The method of claim 1, wherein said scale is customizable to a plurality of aspect ratios.

12. The method of claim 1, further comprising marking at least one region of interest in at least one of said plurality of images.

13. The method of claim 1, wherein said associated quality corresponds to a specified acceptable quality grade.

14. The method of claim 13, wherein said specified acceptable quality grade varies across said plurality of regions.

15. A method of scanning a patient's body cavity using a tip of a multiple viewing elements endoscope associated with a control unit for executing the method, the method comprising the steps of:
automatically recording a start time corresponding to a beginning of an insertion process of said tip within said body cavity;
acquiring a plurality of images of an internal wall of said body cavity during said insertion process;
identifying at least one anomaly within at least one of said plurality of images;
recording and associating a plurality of information with at least one of said plurality of images in real time, wherein at least one of said plurality of information is a first time stamp corresponding to a time taken by said tip to reach proximate said at least one anomaly during said insertion process;
automatically recording an end time corresponding to an end of said insertion process and a beginning of a withdrawal process of said tip from said body cavity;
automatically recording a second time stamp corresponding to a time elapsed during said withdrawal process; and
generating an alert corresponding to said at least one anomaly when said second time stamp is approximately equal to a difference between said end time and said first time stamp.

16. The method of claim 15, wherein said at least one anomaly is identified by a physician by pressing a button on a handle of said endoscope indicating a location of said tip at said at least one anomaly.

17. The method of claim 15, wherein said plurality of information further comprises at least one of an average color of the internal wall, date and time of said scanning, type, size and anatomical location of said at least one anomaly, type of treatment performed or recommended for future scanning with reference to said at least one anomaly, visual markings to highlight said at least one anomaly, dictation recorded by a physician, and a plurality of patient information including age, gender, weight and body mass index.

18. The method of claim 15, further comprising displaying a bar, said bar being a composite representation of said plurality of images acquired during progress of said insertion process and said plurality of information associated with said at least one of said plurality of images.

19. The method of claim 18, further comprising displaying a two dimensional reference frame corresponding to said body cavity.

20. A method of scanning a patient's body cavity using a tip of a multiple viewing elements endoscope associated with a control unit for executing the method, the method comprising the steps of:
selecting a reference frame, of a shape and a scale, from a plurality of reference frames corresponding to the body cavity and to at least one attribute of the patient, wherein said reference frame comprises a plurality of locations corresponding to a plurality of regions of an internal wall of the body cavity;
automatically recording a start time corresponding to a beginning of an insertion process of said tip within said body cavity;
acquiring a plurality of images during said insertion process, each of said plurality of images corresponding to each of said plurality of regions and accordingly to each of said plurality of locations on said reference frame, and wherein each of said plurality of images has an associated quality;
identifying at least one anomaly within at least one of said plurality of images;
recording and associating a plurality of information with at least one of said plurality of images in real time, wherein at least one of said plurality of information is a first time stamp corresponding to a time taken by said tip to reach proximate said at least one anomaly during said insertion process;
mapping, in real time, each of said plurality of images to corresponding each of said plurality of locations on said reference frame, wherein said mapping is done in a sequence in which said plurality of images are acquired and wherein said mapping includes said plurality of information;
automatically recording an end time corresponding to an end of said insertion process and a beginning of a withdrawal process of said tip from said body cavity;
automatically recording a second time stamp corresponding to a time elapsed during said withdrawal process; and
generating an alert corresponding to said at least one anomaly when said second time stamp is approximately equal to a difference between said end time and said first time stamp.

21. The method of claim 20, wherein said plurality of information further comprises at least one of an average color of the internal wall, date and time of said scanning, type, size and anatomical location of said at least one anomaly, type of treatment performed or recommended for future scanning with reference to said at least one anomaly, visual markings to highlight said at least one anomaly, dictation recorded by a physician, and a plurality of patient information including age, gender, weight and body mass index.

22. The method of claim 20, wherein said acquisition of said plurality of images continues until all locations of said plurality of locations of said reference frame are mapped.

23. The method of claim 20, further comprising automatically replacing an image from said plurality of images with an alternative image if an associated quality of said image is lower than an associated quality of said alternative image, wherein said image and said alternative image both correspond to a same region from said plurality of regions.
